SEQUENCE LISTING (1 sequence)

SEQ ID NO: 1 - DNA, 1303 bp, Homo sapiens
gtccaggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcgactcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

(19) United States
(12) Patent Application Publication
(10) Pub. No.: US 2006/0052322 A1
(43) Pub. Date: Mar. 9, 2006

(54) Combination treatment of cancer with elicitor of gene product expression and gene-product targeting agent

(75) Inventors: Jack A. Roth, Houston, TX (US); Guido Schumacher, Berlin (DE); Sunil Chada, Missouri City, TX (US)

Correspondence Address: FULBRIGHT & JAWORSKI, LLP, 1301 MCKINNEY, SUITE 5100, HOUSTON, TX, 77010-3095, US

(73) Assignee: INTROGEN THERAPEUTICS, INC.

(21) Appl. No.: 11/150,521

(22) Filed: Jun. 10, 2005

Related U.S. Application Data
(60) Provisional application No. 60/579,036, filed on Jun. 11, 2004.

(51) Int. Cl. A61K 48/00

(52) U.S. Cl. 514/44

(57) ABSTRACT

The present invention concerns cancer therapy employing an expression construct that affects regulation of one or more particular nucleic acid sequences that encodes a gene product to which an agent is then targeted. In specific embodiments, the present invention relates to the use of p53 gene therapy to treat cancers in combination with Erbitux™(cetuximab). Viral and non-viral gene delivery systems are disclosed.

The present invention claims priority to U.S. Provisional Patent Application Ser. No. 60/579,036, filed Jun. 11, 2004, which is incorporated by reference herein in its entirety.

### BACKGROUND OF THE INVENTION

I. Field of the Invention

The present invention relates generally to at least the fields of oncology, pathology, cell biology, molecular biology and gene therapy. More particularly, it concerns the use of combination therapy with a composition that regulates expression of a particular gene product and a composition that targets the gene product.

II. Description of Related Art

Cancer is a leading cause of death in most countries, and the result of billions of dollars in healthcare expense around the world. Through great effort, significant advances have been made in treating cancer, primarily due to the development of radiation and chemotherapy-based treatments. Unfortunately, a common problem is tumor cell resistance to radiation and chemotherapeutic drugs. For example, NSCLC accounts for at least 80% of the cases of lung cancer, but patients with NSCLC are generally unresponsive to chemotherapy (Doyle, 1993). One goal of current cancer research is to find ways to improve the efficacy of these “traditional” therapeutic regimens, and the genetics of cancer cells has led to dramatic discoveries and a greater understanding of disease development.

It is now well-established that a variety of cancers are caused, at least in part, by genetic abnormalities that result in either the overexpression of cancer causing genes, called “oncogenes,” or from loss of function mutation in protective genes, often called “tumor suppressor” genes. An important gene of the latter category is p53—a 53 kD nuclear phosphoprotein that controls cell proliferation. Mutations to the p53 gene and allele loss on chromosome 17p, where this gene is located, are among the most frequent alterations identified in human malignancies. The p53 protein is highly conserved through evolution and is expressed in most normal tissues. Wild-type p53 has been shown to be involved in control of the cell cycle (Mercer, 1992), transcriptional regulation (Fields and Jang, 1990; Mietz et al., 1992), DNA replication (Wilcock and Lane, 1991; Bargonetti et al., 1991), and induction of apoptosis (Yonish-Rouach et al., 1991; Shaw et al., 1992).

Various mutant p53 alleles are known in which a single base substitution results in the synthesis of proteins that have quite different growth regulatory properties and, ultimately, lead to malignancies (Hollstein et al., 1991). In fact, the p53 gene has been found to be the most frequently mutated gene in common human cancers (Hollstein et al., 1991; Weinberg, 1991), and is particularly associated with those cancers linked to cigarette smoke (Hollstein et al., 1991; Zakut-Houri et al., 1985). The overexpression of p53 in breast tumors has also been documented (Casey et al., 1991). Interestingly, however, the beneficial effect of p53 are not limited to cancers that contain mutated p53 molecules. In a series of papers, Clayman et al. (1994; 1995a; 1995b) demonstrated that growth of cancer cells expressing wild-type p53 molecules was nonetheless inhibited by expression of p53 from a viral vector.

As a result of these findings, considerable effort has been placed into p53 gene therapy. Retroviral delivery of p53 to humans was reported some time ago (Roth et al., 1996). There, a retroviral vector containing the wild-type p53 gene under control of a beta-actin promoter was used to mediate transfer of wild-type p53 into 9 human patients with non-small cell lung cancers by direct injection. No clinically significant vector-related toxic effects were noted up to five months after treatment. In situ hybridization and DNA polymerase chain reaction showed vector-p53 sequences in post-treatment biopsies. Apoptosis (programmed cell death) was more frequent in post-treatment biopsies than in pretreatment biopsies. Tumor regression was noted in three patients, and tumor growth stabilized in three other patients. Similar studies have been conducted using adenovirus to deliver p53 to human patients with squamous cell carcinoma of the head and neck (SCCHN) (Clayman et al., 1998). Surgical and gene transfer-related morbidities were minimal, and the overall results provided preliminary support for the use of Ad-p53 gene transfer as a surgical adjuvant in patients with advanced SCCHN.

Theodorescu et al. (1991) describe upregulation of epidermal EGFR in transitional cell carcinoma cells that overexpress the c-Ha-ras gene.

Despite these successes, there remains a need to identify specific patient subsets that will most benefit from these procedures, and as a corollary, to identify methods which improve the chance of clinical benefit to these patients. One way in which cancer therapies may be improved is with the combination of multiple anti-cancer therapies. There are numerous examples of drugs and biologicals that, even though efficacious as individual therapies, show greatly improved clinical benefit when provided in combinations. However, it is rarely if ever clear, a priori, which combinations will provide such clinical benefits.

### SUMMARY OF THE INVENTION

Thus, in accordance with the present invention, there are methods and compositions concerning treating cancer with combination therapy by delivering to one or more cancer cells at least two agents. Specifically, the combination therapy includes a first composition, which is a gene therapy composition, that directly or indirectly affects gene expression of a particular gene product in addition to a second composition that targets the particular gene product, thereby evoking at least a reduction in proliferation of the cancer cell and, preferably, death of the cancer cell. That is, the present inventors have identified certain types of gene therapies that not only provide therapy to a cancer cell themselves but also elicit expression of another useful target for therapy. In particular embodiments, upon delivery of gene therapy to a cell, other targets become activated in the cell, and the present invention further directs a second therapeutic composition to these targets in addition to the gene therapy composition itself.

In particular embodiments, this may be further defined as a method of treating a subject with cancer comprising administering to the subject a p53 expression construct and an agent that targets the expressed product of a p53 expression construct-responsive nucleic acid sequence in the cell. In specific embodiments, the expression construct and agent are provided in amounts that treat the cancer. In further specific embodiments, the p53 expression construct is further defined as comprising a nucleic acid segment encoding p53, said segment under the control of a promoter active in a cancer cell of the subject, wherein the p53 expression construct expresses p53 in the cancer cell. In specific embodiments, the agent is further defined as targeting the expressed product of a p53 expression construct-responsive nucleic acid sequence or a downstream target thereof.

In particular embodiments of the invention, the p53 expression construct upregulates expression of the p53 expression construct-responsive nucleic acid sequence. In other embodiments, the p53 expression construct down-regulates expression of the p53 expression construct-responsive nucleic acid sequence. In further embodiments, the agent targets a downstream gene product of the expressed product from a p53 expression construct-responsive nucleic acid sequence. For example, the p53 expression construct may downregulate expression of a particular p53 expression construct-responsive nucleic acid sequence, and this downregulation may lead to upregulation of a nucleic acid sequence downstream therefrom, wherein the agent targets the gene product of this upregulated downstream sequence. A skilled artisan recognizes that expression of different targets may be affected differentially with, for example, Ad-p53 compared to p53 in another vector or alone.

In particular embodiments, the first composition that directly or indirectly affects gene expression comprises a tumor suppressor, a cytokine, a transcription factor, or a combination or mixture thereof. More specifically, the first composition may comprise p53, mda-7, or fus-1, in exemplary embodiments. The first composition may be delivered as a polypeptide or as a polynucleotide encoding at least part of the polypeptide, and any suitable delivery elements may be employed. In specific embodiments, the first composition is delivered comprised with or on a vector, such as a viral vector, for example an adenoviral vector, or a non-viral vector, including a plasmid.

In a purely exemplary embodiment, Ad-p53 is delivered to a cancer cell and upregulates expression of EGFR. The upregulation results in an increased number of EGFR molecules on the surface of the cell. The cell is subsequently or concomitantly delivered an agent that targets EGFR, thereby resulting directly or indirectly in death of the cell or at least a reduction in its proliferation, such as, for example, by blocking its tyrosine kinase activity at least partially. In specific embodiments, the agent is an antibody that targets EGFR, which may then render it non-functional, and results in death of the cell. Such an agent that targets EGFR may comprise such as Erbitux™(cetuximab), Iressa™(gefitinib), Tarceva® (erlotinib), or a combination or mixture thereof.

In particular aspects, the manner of the invention is such that therapy with the combination described herein is synergistic in nature, although in alternative embodiments the combination provides additive effects for therapy.

Although in particular embodiments the first composition is a p53 expression construct, such as an Ad-p53 expression construct, in other embodiments the first composition is not a p53-related construct. That is, one of skill in the art recognizes that the invention may be employed with expression constructs that upregulate and/or downregulate particular responsive nucleic acid sequences thereto, and these responsive nucleic acid sequences or the gene products thereof are then targeted by an agent. Although any expression construct would be suitable in the invention so long as it indirectly or directly provides a target for a second composition that then provides a means for destruction or reduction of proliferation of the cell, such suitable compositions may be determined by one of skill in the art utilizing standard reagents and methods. For example, an expression profile may be determined in one or more particular cancer cells, and there may be identification of a nucleic acid sequence that regulates expression of at least one nucleic acid sequence for which there is or may be a suitable target agent. In particular embodiments, the expression profile is determined in one or more cancer cells that are refractory to one or more particular cancer treatments.

In an additional embodiment of the present invention, there is an assay to determine useful combinations of the invention. For example, there is an assay provided herein to identify one or more compositions that elicit alterations in expression, such as upregulation of expression, and this may be performed in a cell of interest, such as a cancer cell that is at least similar to the cell to which the ultimate therapy will be applied (such as sharing genotypes, phenotypes, or both). In embodiments wherein a therapy is determined for a chemoresistant cancer, for example, the cell for the assay may be that of a chemoresistant cell. That is, in specific aspects of the invention, there is provided a breast cancer cell to which an agent is delivered, and the agent may be an adenovirus comprising sequence of a particular gene capable of effecting gene regulation. Following delivery, a variety of genes are assayed for changes in expression elicited thereby. This aspect of the assay may comprise microarray analysis, for example. One or more of the upregulated genes are identified and, if determined to encode a target suitable for a drug, it is employed in the second step of the assay, wherein the drug is applied to the cell bearing the upregulated gene, and the biological outcome for the cell is determined. That is, death of the cell, or at least inhibition of its proliferation, identifies the drug and the corresponding agent in this assay as a suitable combination therapy.

A skilled artisan recognizes that the form of the agent for the assay may be considered. For example, an Ad-p53 composition may upregulate/downregulate expression for a profile of genes that for a p53 composition alone may be different. It is well within the skill of the art, for example, to deliver a retroviral-p53 composition to a cancer cell, determine the nucleic acid(s) having alterations in gene expression, identify one or more nucleic acids having expressed gene products of which may be then targeted by a suitable agent, and identify an agent that suitably targets the one or more corresponding expressed gene products.

In alternative embodiments, in lieu of a particular expression construct affecting regulation of a nucleic acid sequence, the invention instead employs radiation to effect upregulation or down regulation of one or more nucleic acid sequences, the expressed gene products of which are then targeted by a suitable agent. Other suitable alternative methods of effecting regulation of a targetable nucleic acid sequence or gene product thereof are contemplated.

In another aspect of the invention, delivery of the first composition results indirectly or directly in alteration of expression of a responsive nucleic acid sequence, such as, for example, growth factor receptors, receptor tyrosine kinases, and other druggable targets, such as those that facilitate combinatorial strategies. However, in particular embodiments the first composition directly upregulates expression of a gene product that is a target for the second composition, which may be referred to as an agent. In particular, upregulation of gene expression results in an increased number of gene product molecules that are then more easily targeted by the second composition. For example, the upregulated gene product may be one that is secreted or that is a cell surface receptor, thereby increasing the numbers of gene products per cell for targeting by the drug. In some aspects of the invention, such as when the upregulated product is a receptor, the agent may affect the receptor itself and/or a signal resulting therefrom. In a particular embodiment, the second composition is an antibody, small molecule, or a mixture or combination thereof. In a further specific embodiment, the second composition is a drug that targets, for example, the EGF receptor, the VEGF receptor, the HER2/neu receptor, and so forth.

In particular embodiments of the invention, there is a method of treating a subject with cancer comprising administering to said subject: (a) a p53 expression construct comprising a nucleic acid segment encoding p53, said segment under the control of a promoter active in a cancer cell of said subject, said p53 expression construct expressing p53 in said cancer cell; and (b) an agent that targets the expressed product of a p53 expression construct-responsive nucleic acid sequence in said cell, whereby said expression construct and agent are provided in amounts that treat said cancer. The expression construct and the agent may be administered concomitantly or in succession, and in specific aspects of the invention the expression construct is administered prior to the agent.

In particular embodiments, the agent is further defined as targeting the expressed product of a p53 expression construct-responsive nucleic acid sequence or a downstream target thereof. The p53-responsive nucleic acid sequence may be upregulated in response to the p53 expression construct, or the p53-responsive nucleic acid sequence may be downregulated in response to the p53 expression construct. In specific embodiments, the p53-responsive nucleic acid sequence encodes a growth factor receptor, a receptor tyrosine kinase, a cell surface receptor, or a combination thereof.

In a particular embodiment, there is provided a method of treating a subject with cancer comprising administering to the subject, in combination, (a) an expression construct comprising a nucleic acid segment encoding p53, the segment under the control of a promoter active in a cancer cell of the subject, the expression construct expressing p53 in the cancer cell; and (b) Erbitux™, whereby the expression construct and Erbitux™ are provided in amounts that treat the cancer.

The expression construct may be a viral expression construct, such as a retroviral construct, a herpesviral construct, an adenoviral construct, an adeno-associated viral construct, or a vaccinia viral construct, including both replication-competent and replication-defective vectors. The expression construct also may be a non-viral expression construct, such as one comprised within a lipid vehicle.

The promoter may be selected from CMV IE, RSV LTR, β-actin, Ad-E1, Ad-E2 or Ad-MLP. The expression vector may also comprises other elements such as an origin of replication, a selectable marker, a polyadenylation signal operably linked to the p53-coding region, a transcription termination signal, a second coding region for a second anti-cancer gene (inducer of apoptosis, toxin, tumor suppressor) and/or an IRES.

It is contemplated that Erbitux™, the exemplary embodiment of the agent that targets a particular exemplary gene product, may be given to the subject within, within at least, or within at most, 5, 10, 15, 20, 25, 30, 35, 40, 45, 50, 55 minutes or 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 86, 84, 90, 96, 102, 108, 114, 120, 126, 130, 136, 142 hours, or 1, 2, 3, 4, 5, 6, 7 days, or 1, 2, 3, 4, 5, 6, 7, 8, 9, 10, 11, 12 weeks, or 4, 5, 6, 7, 8, 10, 12 months or more, or a combination thereof, of the time that the subject is administered a dose of the p53 expression vector.

Cancers that may be treated by methods and compositions of the invention include those of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodernal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Compositions may be administered to a cell or a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation (e.g., aerosol inhalation), by injection, by infusion, by continuous infusion, by localized perfusion bathing target cells directly, via a catheter, via a lavage, in a creme, or in a lipid composition.

The cancer may be metastatic, recurrent, recurrent at a primary tumor site or a metastatic site. The subject may have had surgical resection prior to administration of (a) and/or (b), or following administration (a) and/or (b). Administration (a) may be selected from the group consisting of intratumoral, to a tumor vasculature, local to a tumor, regional to a tumor, and systemic, administration (b) may be selected from the group consisting of intratumoral, to a tumor vasculature, local to a tumor, regional to a tumor, and systemic. Administrations (a) and (b) may be the same or different. The subject may be a human subject.

The method may further comprise an additional distinct cancer therapy, such as chemotherapy, radiotherapy, non-p53 gene therapy, non-Erbitux™ immunotherapy, hormonal therapy, or toxin therapy.

In another embodiment, there is provided a pharmaceutical formulation comprising (a) an expression construct that affects expression of a particular nucleic acid sequence; and (b) an agent that targets the expressed product of the particular nucleic acid sequence.

In another embodiment, there is provided a pharmaceutical formulation comprising (a) an expression construct comprising a nucleic acid segment encoding p53, said segment under the control of a promoter active in a cancer cell of said subject; and (b) Erbitux™, Iressa® (gefitinib) or Tarceva® (erlotinib).

In another embodiment, there is provided a kit comprising, in separate containers, (a) an expression construct that affects expression of a particular nucleic acid sequence; and (b) an agent that targets the expressed product of the particular nucleic acid sequence.

In yet another embodiment, there is provided a kit comprising, in separate containers, (a) an expression construct comprising a nucleic acid segment encoding p53, the segment under the control of a promoter active in a cancer cell of the subject; and (b) Erbitux™.

In additional embodiments of the invention, the p53-responsive nucleic acid sequence is epidermal growth factor receptor (EGFR), cox-2, Bax, IGFBP3, IGFBP4, Mdm2, MMP-2, TGF-alpha, IL-8R, p21, caveolin, DR5, Jun-B, Gpc, HO1, Btk, Htk, Bax, PIG3, Mdm2, PET-3, PET-7, ATDC, B61, PET-1, PET-8, PET-4, Gadd45, RTP, IGBP3, ET2, Mat8, PET-9, Fas, PET-11, 14-3-3σ, endothelin 2, negative growth regulator MyD118, TRAIL receptor 2 (KILLER/DR5), TGF-beta superfamily protein, caveolin, collagen, type II alpha1, nuclear matrix protein NRP/B(NRPB), p53, GADD45, regulator of G-protein signaling 2 G0S8, potassium channel alpha subunit, tyrosine protein kinase receptor eck, Pig3, actin alpha 2, APO-1/FAS, cystathionin-beta-synthase, macrophage stimulating protein (msp), complement component 4A, tetraspan NET-1, keratin 5, plasminogen activator inhibitor PAI-1, Ral GDP dissociation stimulator, collagen type VI alpha 1, TGF-alpha, endogenous retrovirus H and E sequence, keratin 17, EST similar to KIAA0835 protein, keratin 19, rhoHP 1, serum amyloid a protein precursor, interferon-induced 17-kD protein, interleukin-2 receptor beta chain (IL-2Rb), annexin-XIII, semaphorin V, neutrophil NADPH oxidase 2, estradiol 17 beta dehydrogenase 1, BMP4, P protein (melanocyte-specific transporter), thrombospondin 1, human activated p21cdc42Hs kinase (ack), P2XM, Pig12, phosphoglyverate mutase I, possible GTP-binding protein hsr1, superoxide dismutase 3 (extracellular), cyclin-dependent kinase 2, retinoblastoma-binding protein, DNA replication licensing factor cdc47 homolog, prothymosin alpha, EST similar to cyclin B2, ADP-ribosylation factor-like protein 2 (ARL2), human non-histone chromosomal protein HMG-17, mitotic feedback control protein Madp2 homology, KIAA0101 gene, HMG2, KIAA0030, prostatic binding protein, ATPase Na+/K+ transporting beta 1 polypeptide, lamin B receptor, DNA primase polypeptide 1, topoisomerase (DNA) II alpha, Myb proto-oncogene protein, human effector cell protease receptor-1 (EPR-1), CCAAT/enhancer binding protein C/EPBalpha, ribosomal protein S6 kinase 90 kD polypeptide 2, and InsP3 5 phosphatase, component C1 inhibitor, catechol o-methyltransferase, L-histidine decarboxylase, carboxyl ester lipase, interleukin 8 receptor-alpha, alpha-fetoprotein, alpha 1-acid glyprotein 2, cardiotrophin, IGFBP4, JunB, myoglobin, or MDR1, for example.

In an embodiment of the present invention, there is a method of treating a subject with cancer comprising administering to the subject: (a) a p53 expression construct comprising a nucleic acid segment encoding p53, the segment under the control of a promoter active in a cancer cell of the subject, the p53 expression construct expressing p53 in t cancer cell; and (b) an agent that targets the expressed product of a p53 expression construct-responsive nucleic acid sequence in said cell, whereby the expression construct and agent are provided in amounts that treat said cancer. In a specific embodiment, the expression construct and the agent are administered concomitantly or in succession, such as wherein the expression construct is administered prior to the agent. The method may be further defined as indirectly or directly producing apoptosis in said cancer cell. The method may also be further defined as conferring chemosensitivity to said cancer cell.

In particular aspects, the agent is further defined as targeting the expressed product of a p53 expression construct-responsive nucleic acid sequence or a downstream nucleic acid sequence therefrom. In a specific embodiment, the p53-responsive nucleic acid sequence is upregulated in response to the p53 expression construct. In another specific embodiment, the p53-responsive nucleic acid sequence is downregulated in response to the p53 expression construct. In certain aspects, the p53-responsive nucleic acid sequence encodes a growth factor receptor, a receptor tyrosine kinase, a cell surface receptor, or a combination thereof.

Specific examples of p53-responsive nucleic acid sequences include epidermal growth factor receptor (EGFR), cox-2, Bax, IGFBP3, IGFBP4, Mdm2, MMP-2, TGF-alpha, IL-8R, p21, caveolin, DR5, Jun-B, Gpc, HO1, Btk, Htk, Bax, PIG3, Mdm2, PET-3, PET-7, ATDC, B61, PET-1, PET-8, PET-4, Gadd45, RTP, IGBP3, ET2, Mat8, PET-9, Fas, PET-11, 14-3-3σ, endothelin 2, negative growth regulator MyD118, TRAIL receptor 2 (KILLER/DR5), TGF-beta superfamily protein, caveolin, collagen, type II alpha1, nuclear matrix protein NRP/B(NRPB), p53, GADD45, regulator of G-protein signaling 2 G0S8, potassium channel alpha subunit, tyrosine protein kinase receptor eck, Pig3, actin alpha 2, APO-1/FAS, cystathionin-beta-synthase, macrophage stimulating protein (msp), complement component 4A, tetraspan NET-1, keratin 5, plasminogen activator inhibitor PAI-1, Ral GDP dissociation stimulator, collagen type VI alpha 1, TGF-alpha, endogenous retrovirus H and E sequence, keratin 17, EST similar to KIAA0835 protein, keratin 19, rhoHP 1, serum amyloid a protein precursor, interferon-induced 17-kD protein, interleukin-2 receptor beta chain (IL-2Rb), annexin-XIII, semaphorin V, neutrophil NADPH oxidase 2, estradiol 17 beta dehydrogenase 1, BMP4, P protein (melanocyte-specific transporter), thrombospondin 1, human activated p21cdc42Hs kinase (ack), P2XM, Pig12, phosphoglyverate mutase I, possible GTP-binding protein hsr1, superoxide dismutase 3 (extracellular), cyclin-dependent kinase 2, retinoblastoma-binding protein, DNA replication licensing factor cdc47 homolog, prothymosin alpha, EST similar to cyclin B2, ADP-ribosylation factor-like protein 2 (ARL2), human non-histone chromosomal protein HMG-17, mitotic feedback control protein Madp2 homology, KIAA0101 gene, HMG2, KIAA0030, prostatic binding protein, ATPase Na+/K+ transporting beta 1 polypeptide, lamin B receptor, DNA primase polypeptide 1, topoisomerase (DNA) II alpha, Myb proto-oncogene protein, human effector cell protease receptor-1 (EPR-1), CCAAT/enhancer binding protein C/EPBalpha, ribosomal protein S6 kinase 90 kD polypeptide 2, and InsP3 5 phosphatase, component C1 inhibitor, catechol o-methyltransferase, L-histidine decarboxylase, carboxyl ester lipase, interleukin 8 receptor-alpha, alpha-fetoprotein, alpha 1-acid glyprotein 2, cardiotrophin, IGFBP4, JunB, myoglobin, or MDR1.

In particular aspects of the invention the agent is a small molecule, an antibody, such as a monoclonal antibody. In another aspect, the p53-responsive nucleic acid sequence is EGFR and the agent is Erbitux™, gefitinib, or erlotinib.

Expression constructs of the invention may include a viral expression construct, such as a retroviral construct, a herpesviral construct, an adenoviral construct, an adeno-associated viral construct, or a vaccinia viral construct. The viral expression construct may be a replication-competent virus or a replication-defective virus. In a specific embodiment, the expression construct is a non-viral expression construct, such as one that is comprised within a lipid vehicle. Promoters utilized in the invention may be selected from CMV IE, RSV LTR, β-actin, Ad-E1, Ad-E2 or Ad-MLP.

In another embodiment, there is a method of treating a subject with cancer comprising administering to the subject, in combination, (a) an expression construct comprising a nucleic acid segment encoding p53, the segment under the control of a promoter active in a cancer cell of the subject, the expression construct expressing p53 in the cancer cell; and (b) Erbitux™, whereby said expression construct and Erbitux™ are provided in amounts that treat the cancer. In specific embodiments, the cancer is selected from the group consisting of brain cancer, head & neck cancer, esophageal cancer, tracheal cancer, lung cancer, liver cancer stomach cancer, colon cancer, pancreatic cancer, breast cancer, cervical cancer, uterine cancer, bladder cancer, prostate cancer, testicular cancer, skin cancer, rectal cancer lymphoma and leukemia.

In a specific embodiment, the time period between administration (a) and (b) is less than about 4 hours, is less than about 12 hours, is about 1 day, is about 2 days, is about 7 days, is about 14 days, is about 1 month, or is about 2 months, for example. In certain aspects of the invention, the subject has had surgical resection prior to administration (a), following administration (b), or both. In particular aspects, administration (a), or (b), or both, is selected from the group consisting of intratumoral, to a tumor vasculature, local to a tumor, regional to a tumor, and systemic. In further embodiments, the subject is a human subject and/or the method further comprises an additional distinct cancer therapy, such as chemotherapy, radiotherapy, non-p53 gene therapy, non-Erbitux™ immunotherapy, hormonal therapy, or toxin therapy.

In an additional embodiment, there is a pharmaceutical formulation comprising (a) an expression construct comprising a nucleic acid segment encoding p53, said segment under the control of a promoter active in a cancer cell of said subject; and (b) an agent that targets a gene product of the nucleic acid segment.

In another embodiment, there is a kit comprising, in separate containers, (a) an expression construct comprising a nucleic acid segment encoding p53, said segment under the control of a promoter active in a cancer cell of said subject; and (b) an agent that targets a gene product of the nucleic acid segment.

In another embodiment, there is a pharmaceutical formulation comprising (a) an expression construct comprising a nucleic acid segment encoding p53, said segment under the control of a promoter active in a cancer cell of said subject; and (b) Erbitux™.

In an additional embodiment, there is a kit comprising, in separate containers, (a) an expression construct comprising a nucleic acid segment encoding p53, said segment under the control of a promoter active in a cancer cell of said subject; and (b) Erbitux™.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The term “about” means, in general, the stated value plus or minus 5%.

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The attached drawings form part of the present specification and are included to further demonstrate certain aspects of the present invention. The invention may be better understood by reference to one or more of these drawings in combination with the detailed description of specific embodiments presented herein.

FIGS. 1A-1B show in vitro proliferation assays of MDA-MB-468 (FIG. 1A) and H1299 (FIG. 1B) cells treated with Ad-p53 and Ad-β-gal at a dose of 1 MOI (FIG. 1A) or 0.5 MOI (FIG. 1B). Cetuximab was added to a final concentration of 30 nM. Arrows indicate the time points of treatment. The values shown are the averages of triplicate cell counts. Error bars indicate SEM.

FIGS. 2A-2B demonstrate that EGFR expression is highly induced by p53. After transfection with Ad-p53, the present inventors found a time-dependent upregulation of the EGF receptor (FIG. 2A). In FIG. 2B, the membrane was stripped and reprobed for phosphotyrosine.

FIGS. 3A-3B show p53 expression as it relates to p21 w The p53-mutated cell line MDA-MB-468 was transduced with Ad-FLAGp53, and the expression of FLAGp53 was measured over a five-day period by western blot (FIG. 3A). Expression levels of p21^{WAF/CIP1} were also strongly induced by p53. FIG. 3B shows similar results for the H1299 cell line.

FIGS. 4A-4B show western blot analysis of p27 expression in MDA-MB-468 (FIG. 4A) and H1299 cells (FIG. 4B) treated as described herein. The number of days after the first Ad-p53 transduction is shown in parentheses. Numbers under the blots indicate the level of upregulation in treated cells compared with control untreated cells.

FIGS. 5A-5F show that overexpression of p27^{KIP1} induces apoptosis. FIGS. 5A-5F show the different treatment groups of H1299 cells in TUNEL staining.

FIGS. 6A-6B show growth inhibition of subcutaneous tumors in nude mice. Ad-p53 was injected intratumorally and C225 intraperitoneally. The tumor size shown for each group is the average of 5 tumors. Error bars show SEM.

### DESCRIPTION OF THE ILLUSTRATIVE EMBODIMENTS

I. The Present Invention

One approach to cancer therapy employs combination of two embodiments to reduce proliferation and/or destroy one or more cancer cells. The combinatorial approach provides a dual mechanism to affect the cell, which is particularly useful for treatment of cancer and, in particular, resistant cancer cells. The present invention provides a bipartite approach to cancer treatment, which in specific embodiments may be considered additive and in other embodiments may be considered synergistic.

Specifically, the present invention encompasses the delivery to a cancer cell of an expression construct that targets one or more nucleic acid sequences, and the gene product that results from the expression of the one or more nucleic acid sequence(s) is then targeted by an agent, such as a drug, including a small molecule or antibody, for example. The cell is thereby reduced in proliferation or destroyed. In other words, a therapeutic expression construct is delivered to a cell and affects expression of one or more nucleotide sequences, and the gene product encoded by the nucleotide sequence is then targeted by an agent.

In specific embodiments, the cell has reduced proliferation or is destroyed by the action of both a therapeutic expression construct and a therapeutic agent that targets a gene product encoded by a nucleotide sequence the expression of which is affected by the expression construct. In particular embodiments, the expression of one or more particular nucleic acid sequences is upregulated by the expression construct, thereby generating an increased level of gene product molecules from the upregulated nucleic acid sequence. This increased level of molecules provides more targets for the agent, thereby enhancing the ability to reduce the proliferation of or destroy the cancer cell. Thus, in particular embodiments it is the combination of the two compositions that affects the cancer cell.

In particular embodiments, the expression construct comprises an adenoviral p53 composition, and in specific embodiments the nucleic acid sequences responsive to a p53 expression construct comprise a p53-binding motif comprising two copies of the following sequence separated by 0 to about 13 base pairs: 5′-PuPuPuC(A/T)(T/A)GPyPyPy-3′.

Embodiments of the present invention may be described herein with the non-limiting specific embodiments of Ad-p53 upregulating expression of EGFR, which is then targeted with Erbitux™, Iressa® (gefitinib) or Tarceva®.

As discussed above, p53 gene therapy at the clinical level has been under study for a decade. Overall, the success of this approach has been remarkable, showing substantial increased benefits over that seen with traditional therapeutic approaches. Moreover, the side effects of gene therapy appear minimal, and there have been no confirmed deaths associated with the therapy. However, as with most anti-cancer treatments, there still remains a substantial need to improve the efficacy of p53 gene therapy.

One way of improving a given cancer therapy is to attempt to identify other therapies that work in combination with a selected cancer therapy. p53 is no different, with several chemo- and radiotherapies having been shown to work advantageously in combination with retroviral and adenoviral delivery of p53. The present invention provides another aspect of such combinations therapies, employing the anti-EGFR antibody Erbitux™, for example. Further benefit may also be obtained by additional treatment with at least one round of radiation, other chemotherapy, surgery, and so forth. Together, these particular treatment combinations provide increased clinical benefits over comparable monotherapies.

The Erbitux™ therapy that is provided in combination with p53 gene therapy may occur contemporaneously with the p53 gene therapy, although both earlier and later time points are contemplated. The present invention may be utilized in a variety of cancers, including sarcomas and carcinomas, and in particular, lymphomas, leukemias, gliomas, adenocarcinomas, squamous cell carcinomas (including head and neck), non-small cell cancer (including lung), melanomas, and others.

Delivery of the p53 expression constructs, such as Advexin™ and/or Erbitux™ and/or radiation or other chemotherapy to patients is contemplated through a variety of different routes, using a variety of different regimens, and include local (intratumoral, tumor vasculature), regional and systemic delivery, for example. Exemplary regimens for delivery of p53 gene therapy may follow those described in the examples, but more generally will involve one, two, three, four, five, six or more administrations of the p53 expression vector. Similarly, Erbitux™, radio- or chemotherapy may be provided in multiple administrations, in certain aspects.

The details for practicing the present invention are provided in the following pages, and the embodiments of Ad-p53 used in conjunction with Erbitux™ is provided merely as an illustrative non-limiting embodiment.

II. Expression Constructs of the Invention

The present invention employs expression constructs that themselves affect the expression of one or more particular nucleic acid sequences, wherein the sequence encodes a targetable gene product. Any suitable expression construct may be utilized in the invention, so long as the construct affects expression of at least one nucleic acid sequence that encodes a targetable gene product. In specific embodiments, the expression construct upregulates expression of one or more nucleic acid sequences that encode a targetable gene product. However, in some embodiments the same expression construct may upregulate expression of some nucleic acid sequences while downregulating expression or not affecting expression of other nucleic acid sequences.

A skilled artisan recognizes standard means in the art of identifying particular nucleic acid sequences that encode targetable gene products, including by sequencing at least part of the sequences having affected expression or by interpreting gene expression microchips, such as those commercially available from Affymetrix (Santa Clara, Calif.), for example.

Although the expression construct may be of any suitable form, such as a viral vector or a non-viral vector, including a plasmid, in particular embodiments the construct comprises an adenoviral vector.

III. Adenoviral-p53 Expression Construct-Responsive Nucleic Acid Sequences

The present invention utilizes as part of a combinatorial approach to cancer therapy an expression construct that affects expression of a gene product that itself is the target of an agent, such as a drug. Although any suitable expression construct may be employed, in specific embodiments it is an adenoviral p53 expression construct.

The exemplary adenoviral p53 expression constructs affect expression of a variety of nucleic acid sequences, and expression of some nucleic acid sequences may be upregulated whereas others are downregulated. For example, EGFR or cox-2 are upregulated in response to adenovirus-p53. Also, Yu et al. (1999) describe upregulation of a variety of nucleic acid sequences in response to a tTA expression vector harboring a bicistronic transcription unit and a bidirectional tTA-responsive promoter regulating expression of green fluorescent protein from one direction and p53 from the other. Exemplary p53 expression construct responsive nucleic acid sequences identified therein include p21, caveolin, DR5, Jun-B, Gpc, HO1, Btk, Htk, Bax, PIG3, Mdm2, p53 early transcripts 3 (PET-3), PET-7, ATDC, B61, PET-1, PET-8, PET-4, Gadd45, RTP, IGBP3, ET2, Mat8, PET-9, Fas, PET-11, and 14-3-3σ.

Zhao et al. (2000) analyzed p53-regulated gene expression utilizing oligonucleotide arrays, and in specific embodiments the p53 was zinc-induced p53. Exemplary p53-responsive sequences identified therein include the following: endothelin 2, negative growth regulator MyD118, TRAIL receptor 2 (KILLER/DR5), TGF-beta superfamily protein, p21/wafl, caveolin, collagen, type II alpha1, nuclear matrix protein NRP/B(NRPB), p53, GADD45, regulator of G-protein signaling 2 G0S8, potassium channel alpha subunit, tyrosine protein kinase receptor eck, Pig3, actin alpha 2, APO-1/FAS, cystathionin-beta-synthase, macrophage stimulating protein (msp), complement component 4A, tetraspan NET-1, keratin 5, plasminogen activator inhibitor PAI-1, Ral GDP dissociation stimulator, collagen type VI alpha 1, IGFBP-3, 14-3-3σ, TGF-alpha, endogenous retrovirus H and E sequence, keratin 17, EST similar to KIAA0835 protein, keratin 19, rhoHP1, serum amyloid a protein precursor, interferon-induced 17-kD protein, interleukin-2 receptor beta chain (IL-2Rb), annexin-XIII, semaphorin V, neutrophil NADPH oxidase 2, estradiol 17 beta dehydrogenase 1, BMP4, P protein (melanoxyte-specific transporter), thrombospondin 1, human activated p21cdc42Hs kinase (ack), P2XM, Pig12, phosphoglyverate mutase I, possible GTP-binding protein hsr1, superoxide dismutase 3 (extracellular), cyclin-dependent kinase 2, retinoblastoma-binding protein, DNA replication licensing factor cdc47 homolog, prothymosin alpha, EST similar to cyclin B2, ADP-ribosylation factor-like protein 2 (ARL2), human non-histone chromosomal protein HMG-17, mitotic feedback control protein Madp2 homology, KIAA0100 gene; HMG2, KIAA0030, prostatic binding protein, ATPase Na+/K+ transporting beta 1 polypeptide, lamin B receptor, DNA primase polypeptide 1, topoisomerase (DNA) II alpha, Myb proto-oncogene protein, human effector cell proease receptor-1 (EPR-1), CCAAT/enhancer binding protein C/EPBalpha, ribosomal protein S6 kinase 90 kD polypeptide 2, and InsP3 5 phosphatase.

Sheikh et al. (1997) concerns identification of an exemplary p53-responsive site in the EGFR promoter residing at position −167/−105.

In Wang et al. (2001), p53 target genes were analyzed employing multiple approaches. Specific sequences highlighted therein include component C1 inhibitor, catechol o-methyltransferase, L-histidine decarboxylase, carboxyl ester lipase, interleukin 8 receptor-alpha, alpha-fetoprotein, alpha 1-acid glyprotein 2, cardiotrophin, IGFBP4, JunB, and myoglobin.

Yu et al. (2004) describe reversal of 5 fluorouracil resistance following delivery of adenoviral p53 in resistant human colon cells, wherein MDR1 gene expression was reduced following delivery. Therefore, in specific embodiments, a p53 expression construct is delivered to a chemoresistant cancer cell, which overcomes uncontrolled upregulation of MDR1 gene expression, thereby helping to restore cell growth inhibition and apoptosis and providing targets for an agent.

IV. Agent Targeting the Nucleic Acid Sequence or Expressed Gene Product Thereof

The present invention employs an expression construct that affects regulation of one or more particular nucleic acid sequences, and the expressed product thereof then becomes a target for an agent.

A skilled artisan recognizes that the agent may be any suitable composition, such that it targets the expressed gene product and that its delivery results in the indirect or direct reduction of proliferation or death of one or more cancer cells. In specific embodiments, the agent is an antibody, such as a monoclonal antibody, for example, against a cell surface receptor. In additional specific embodiments, the agent comprises Erbitux™, which is an antibody against the EGF receptor. In further specific embodiments, the agent comprises Herceptin (Trastuzumab), directed against the HER2/neu gene product. In particular embodiments, monoclonal antibodies target the gene product of the upregulated or downregulated nucleic acid sequence. Examples of monoclonal antibodies for cancer include the following: Rituximab, Trastuxumab, Gemtuzumab ozogamicin, Alemzutumab, Ibritumomab, Iressa, Tarceva, Avastin™, Velcade, or Gleevac.

A skilled artisan recognizes how to identify agents that target the expressed gene product, such as from information obtained in the published literature. Alternatively, the agent may be developed, such as by producing antibodies for the corresponding embodiment or by identifying molecules suitable for binding to the expressed gene product, such as by modeling.

V. p53

p53 is phosphoprotein of about 390 amino acids that can be subdivided into four domains: (i) a highly charged acidic region of about 75-80 residues, (ii) a hydrophobic proline-rich domain (position 80 to 150), (iii) a central region (from 150 to about 300), and (iv) a highly basic C-terminal region. The sequence of p53 is well conserved in vertebrate species, but there have been no proteins homologous to p53 identified in lower eucaryotic organisms. Comparisons of the amino acid sequence of human, African green monkey, golden hamster, rat, chicken, mouse, rainbow trout and Xenopus laevis p53 proteins indicated five blocks of highly conserved regions, which coincide with the mutation clusters found in p53 in human cancers evolution.

Although p53 sequences may be identified in the National Center for Biotechnology Information's GenBank database, an exemplary p53 sequence is provided in SEQ ID NO:1 (GenBank Accession No. M14695).

p53 is located in the nucleus of cells and is very labile. Agents which damage DNA induce p53 to become very stable by a post-translational mechanism, allowing its concentration in the nucleus to increase dramatically. p53 suppresses progression through the cell cycle in response to DNA damage, thereby allowing DNA repair to occur before replicating the genome. Hence, p53 prevents the transmission of damaged genetic information from one cell generation to the next initiates apoptosis if the damage to the cell is severe. Mediators of this effect included Bax, a well-known “inducer of apoptosis.”

As discussed above, mutations in p53 can cause cells to become oncogenically transformed, and transfection studies have shown that p53 acts as a potent transdominant tumor suppressor, able to restore some level of normal growth to cancerous cells in vitro. p53 is a potent transcription factor and once activated, it represses transcription of one set of genes, several of which are involved in stimulating cell growth, while stimulating expression of other genes involved in cell cycle control

VI. p53 Polynucleotides

Certain non-limiting but exemplary embodiments of the present invention concern nucleic acids encoding a p53. In certain aspects, both wild-type and mutant versions of these sequences will be employed. The term “nucleic acid” is well known in the art. A “nucleic acid” as used herein will generally refer to a molecule (i.e., a strand) of DNA, RNA or a derivative or analog thereof, comprising a nucleotide base. A nucleotide base includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleotide bases in length. The term “polynucleotide” refers to at least one molecule of greater than about 100 nucleotide bases in length.

In certain embodiments, a “gene” refers to a nucleic acid that is transcribed. In certain aspects, the gene includes regulatory sequences involved in transcription or message production. In particular embodiments, a gene comprises transcribed sequences that encode for a protein, polypeptide or peptide. As will be understood by those in the art, this functional term “gene” includes genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered nucleic acid segments may express, or may be adapted to express proteins, polypeptides, polypeptide domains, peptides, fusion proteins, mutant polypeptides and/or the like.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid as originally isolated, and does not exclude genes or coding regions later added to the nucleic acid by the hand of man.

A. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. Various mechanisms of oligonucleotide synthesis may be used, such as those methods disclosed in, U.S. Pat. Nos. 4,659,774; 4,816,571; 5,141,813; 5,264,566; 4,959,463; 5,428,148; 5,554,744; 5,574,146; 5,602,244 each of which are incorporated herein by reference.

A non-limiting example of an enzymatically produced nucleic acid include nucleic acids produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

B. Purification of Nucleic Acids

A nucleic acid may be purified on polyacrylamide gels, cesium chloride centrifugation gradients, column chromatography or by any other means known to one of ordinary skill in the art (see for example, Sambrook et al., 2001, incorporated herein by reference). In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells. Methods for isolating nucleic acids (e.g., equilibrium density centrifugation, electrophoretic separation, column chromatography) are well known to those of skill in the art.

VII. Expression of Nucleic Acids

In accordance with the present invention, it will be desirable to produce p53 proteins in a cell. Expression typically requires that appropriate signals be provided in the vectors or expression cassettes, and which include various regulatory elements, such as enhancers/promoters from viral and/or mammalian sources that drive expression of the genes of interest in host cells. Elements designed to optimize messenger RNA stability and translatability in host cells may also be included. Drug selection markers may be incorporated for establishing permanent, stable cell clones.

Viral vectors are selected eukaryotic expression systems. Included are adenoviruses, adeno-associated viruses, retroviruses, herpesviruses, lentivirus and poxviruses including vaccinia viruses and papilloma viruses including SV40. Viral vectors may be replication-defective, conditionally-defective or replication-competent. Also contemplated are non-viral delivery systems, including lipid-based vehicles.

A. Vectors and Expression Constructs

The term “vector” is used to refer to a carrier nucleic acid molecule into which a nucleic acid sequence can be inserted for introduction into a cell where it can be replicated and/or expressed. A nucleic acid sequence can be “exogenous ”or “heterologous” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 2001 and Ausubel et al., 1996, both incorporated herein by reference).

The term “expression vector” refers to any type of genetic construct comprising a nucleic acid coding for a RNA capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well, as described below.

In order to express p53, it is necessary to provide an expression vector. The appropriate nucleic acid can be inserted into an expression vector by standard subcloning techniques. The manipulation of these vectors is well known in the art. Examples of fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

In yet another embodiment, the expression system used is one driven by the baculovirus polyhedron promoter. The gene encoding the protein can be manipulated by standard techniques in order to facilitate cloning into the baculovirus vector. A preferred baculovirus vector is the pBlueBac vector (Invitrogen, Sorrento, Calif.). The vector carrying the gene of interest is transfected into Spodoptera frugiperda (Sf9) cells by standard protocols, and the cells are cultured and processed to produce the recombinant protein. Mammalian cells exposed to baculoviruses become infected and may express the foreign gene only. This way one can transduce all cells and express the gene in dose dependent manner.

There also are a variety of eukaryotic vectors that provide a suitable vehicle in which recombinant polypeptide can be produced. HSV has been used in tissue culture to express a large number of exogenous genes as well as for high level expression of its endogenous genes. For example, the chicken ovalbumin gene has been expressed from HSV using an α promoter. Herz and Roizman (1983). The lacZ gene also has been expressed under a variety of HSV promoters.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose. Tables 1 and 2 list several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (for example, as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
α_{1-Antitrypsin}
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 2

| Element | Inducer |
|---|---|
| MT II | Phorbol Ester (TP A) |
| | Heavy metals |
| MMTV (mouse mammary tumor | Glucocorticoids |
| virus) | |
| β-Interferon | Poly(rI)X |
| | Poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al., 1987).

In various embodiments of the invention, the expression construct may comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides in a gene therapy scenario.

B. Viral Vectors

Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

1. Adenoviral Vectors

a. Virus Characteristics

Adenovirus is a non-enveloped double-stranded DNA virus. The virion consists of a DNA-protein core within a protein capsid. Virions bind to a specific cellular receptor, are endocytosed, and the genome is extruded from endosomes and transported to the nucleus. The genome is about 36 kB, encoding about 36 genes. In the nucleus, the “immediate early” E1A proteins are expressed initially, and these proteins induce expression of the “delayed early” proteins encoded by the E1B, E2, E3, and E4 transcription units. Virions assemble in the nucleus at about 1 day post infection (p.i.), and after 2-3 days the cell lyses and releases progeny virus. Cell lysis is mediated by the E3 11.6K protein, which has been renamed “adenovirus death protein” (ADP).

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

Adenovirus may be any of the 51 different known serotypes or subgroups A-F. Adenovirus type 5 of subgroup C is the human adenovirus about which the most biochemical and genetic information is known, and it has historically been used for most constructions employing adenovirus as a vector. Recombinant adenovirus often is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Viruses used in gene therapy may be either replication-competent or replication-deficient. Generation and propagation of the adenovirus vectors which are replication-deficient depends on a helper cell line, the prototype being 293 cells, prepared by transforming human embryonic kidney cells with Ad5 DNA fragments; this cell line constitutively expresses E1 proteins (Graham et al., 1977). However, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Adenovirus growth and manipulation is known to those of skill in the art, and exhibits broad host range in vitro and in vivo. This group of viruses can be obtained in high titers, e.g., 10⁹-10¹³ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

b. Engineering

As stated above, Ad vectors are based on recombinant Ad's that are either replication-defective or replication-competent. Typical replication-defective Ad vectors lack the E1A and E1B genes (collectively known as E1) and contain in their place an expression cassette consisting of a promoter and pre-mRNA processing signals which drive expression of a foreign gene. These vectors are unable to replicate because they lack the E1A genes required to induce Ad gene expression and DNA replication. In addition, the E3 genes can be deleted because they are not essential for virus replication in cultured cells. It is recognized in the art that replication-defective Ad vectors have several characteristics that make them suboptimal for use in therapy. For example, production of replication-defective vectors requires that they be grown on a complementing cell line that provides the E1A proteins in trans.

Several groups have also proposed using replication-competent Ad vectors for therapeutic use. Replication-competent vectors retain Ad genes essential for replication, and thus do not require complementing cell lines to replicate. Replication-competent Ad vectors lyse cells as a natural part of the life cycle of the vector. An advantage of replication-competent Ad vectors occurs when the vector is engineered to encode and express a foreign protein. Such vectors would be expected to greatly amplify synthesis of the encoded protein in vivo as the vector replicates. For use as anti-cancer agents, replication-competent viral vectors would theoretically be advantageous in that they would replicate and spread throughout the tumor, not just in the initially infected cells as is the case with replication-defective vectors.

Yet another approach is to create viruses that are conditionally-replication competent. Onyx Pharmaceuticals recently reported on adenovirus-based anti-cancer vectors which are replication-deficient in non-neoplastic cells, but which exhibit a replication phenotype in neoplastic cells lacking functional p53 and/or retinoblastoma (pRB) tumor suppressor proteins (U.S. Pat. No. 5,677,178). This phenotype is reportedly accomplished by using recombinant adenoviruses containing a mutation in the E1B region that renders the encoded E1B-55K protein incapable of binding to p53 and/or a mutation(s) in the E1A region which make the encoded E1A protein (p289R or p243R) incapable of binding to pRB and/or p300 and/or p107. E1B-55K has at least two independent functions: it binds and inactivates the tumor suppressor protein p53, and it is required for efficient transport of Ad mRNA from the nucleus. Because these E1B and E1A viral proteins are involved in forcing cells into S-phase, which is required for replication of adenovirus DNA, and because the p53 and pRB proteins block cell cycle progression, the recombinant adenovirus vectors described by Onyx should replicate in cells defective in p53 and/or pRB, which is the case for many cancer cells, but not in cells with wild-type p53 and/or pRB.

Another replication-competent adenovirus vector has the gene for E1B-55K replaced with the herpes simplex virus thymidine kinase gene (Wilder et al., 1999a). The group that constructed this vector reported that the combination of the vector plus gancyclovir showed a therapeutic effect on a human colon cancer in a nude mouse model (Wilder et al., 1999b). However, this vector lacks the gene for ADP, and accordingly, the vector will lyse cells and spread from cell-to-cell less efficiently than an equivalent vector that expresses ADP.

The present inventor has taken advantage of the differential expression of telomerase in dividing cells to create novel adenovirus vectors which overexpress an adenovirus death protein and which are replication-competent in and, preferably, replication-restricted to cells expressing telomerase. Specific embodiments include disrupting E1A's ability to bind p300 and/or members of the Rb family members. Others include Ad vectors lacking expression of at least one E3 protein selected from the group consisting of 6.7K, gp19K, RIDα (also known as 10.4K); RIDβ (also known as 14.5K) and 14.7K. Because wild-type E3 proteins inhibit immune-mediated inflammation and/or apoptosis of Ad-infected cells, a recombinant adenovirus lacking one or more of these E3 proteins may stimulate infiltration of inflammatory and immune cells into a tumor treated with the adenovirus and that this host immune response will aid in destruction of the tumor as well as tumors that have metastasized. A mutation in the E3 region would impair its wild-type function, making the viral-infected cell susceptible to attack by the host's immune system. These viruses are described in detail in U.S. Pat. No. 6,627,190.

Other adenoviral vectors are described in U.S. Pat. Nos. 5,670,488; 5,747,869; 5,932,210; 5,981,225; 6,069,134; 6,136,594; 6,143,290; 6,210,939; 6,296,845; 6,410,010; and 6,511,184; U.S. Publication No. 2002/0028785.

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the methods of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as therapeutic vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

6. Non-Viral Delivery

Lipid-based non-viral formulations provide an alternative to adenoviral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (Templeton et al. 1997; WO 98/07408, incorporated herein by reference). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation is said to form a unique structure termed a “sandwich liposome.” This formulation is reported to “sandwich” DNA between an invaginated bilayer or “vase” structure. Beneficial characteristics of these liposomes include a positive to negative charge or p, colloidal stabilization by cholesterol, two-dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

Liposomes are vesicular structures characterized by a lipid bilayer and an inner aqueous medium. Multilamellar liposomes have multiple lipid layers separated by aqueous medium. They form spontaneously when lipids are suspended in an excess of aqueous solution. The lipid components undergo self-rearrangement before the formation of structures that entrap water and dissolved solutes between the lipid bilayers (Ghosh and Bachhawat, 1991). Lipophilic molecules or molecules with lipophilic regions may also dissolve in or associate with the lipid bilayer.

The liposomes are capable of carrying biologically active nucleic acids, such that the nucleic acids are completely sequestered. The liposome may contain one or more nucleic acids and is administered to a mammalian host to efficiently deliver its contents to a target cell. The liposomes may comprise DOTAP and cholesterol or a cholesterol derivative. In certain embodiments, the ratio of DOTAP to cholesterol, cholesterol derivative or cholesterol mixture is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to 1:3, more preferably 2:1 to 1:2, and most preferably 1:1. In further preferred embodiments, the DOTAP and/or cholesterol concentrations are about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, or 30 mM. The DOTAP and/or Cholesterol concentration can be between about 1 mM to about 20 mM, 1 mM to about 18 mM, 1 mM to about 16 mM, about 1 mM to about 14 mM, about 1 mM to about 12 mM, about 1 mM to about 10 mM, 1 to 8 mM, more preferably 2 to 7 mM, still more preferably 3 to 6 mM and most preferably 4 to 5 mM. Cholesterol derivatives may be readily substituted for the cholesterol or mixed with the cholesterol in the present invention. Many cholesterol derivatives are known to the skilled artisan. Examples include but are not limited to cholesterol acetate and cholesterol oleate. A cholesterol mixture refers to a composition that contains at least one cholesterol or cholesterol derivative.

The formulation may also be extruded using a membrane or filter, and this may be performed multiple times. Such techniques are well-known to those of skill in the art, for example in Martin (1990). Extrusion may be performed to homogenize the formulation or limit its size. A contemplated method for preparing liposomes in certain embodiments is heating, sonicating, and sequential extrusion of the lipids through filters of decreasing pore size, thereby resulting in the formation of small, stable liposome structures. This preparation produces liposomal complexes or liposomes only of appropriate and uniform size, which are structurally stable and produce maximal activity.

For example, it is contemplated in certain embodiments of the present invention that DOTAP:Cholesterol liposomes are prepared by the methods of Templeton et al. (1997; incorporated herein by reference). Thus, in one embodiment, DOTAP (cationic lipid) is mixed with cholesterol (neutral lipid) at equimolar concentrations. This mixture of powdered lipids is then dissolved with chloroform, the solution dried to a thin film and the film hydrated in water containing 5% dextrose (w/v) to give a final concentration of 20 mM DOTAP and 20 mM cholesterol. The hydrated lipid film is rotated in a 50° C. water bath for 45 minutes, then at 35° C. for an additional 10 minutes and left standing at room temperature overnight. The following day the mixture is sonicated for 5 minutes at 50° C. The sonicated mixture is transferred to a tube and heated for 10 minutes at 50° C. This mixture is sequentially extruded through syringe filters of decreasing pore size (1 μm, 0.45 μm, 0.2 μm, 0.1 μm).

It also is contemplated that other liposome formulations and methods of preparation may be combined to impart desired DOTAP:Cholesterol liposome characteristics. Alternate methods of preparing lipid-based formulations for nucleic acid delivery are described by Saravolac et al. (WO 99/18933). Detailed are methods in which lipids compositions are formulated specifically to encapsulate nucleic acids. In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

C. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989; Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); and any combination of such methods.

D. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that p53 may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

The nucleotide and protein sequences for p53 have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified and/or expressed using the techniques disclosed herein or by any technique that would be known to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

E. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

VIII. Therapeutic Intervention

In accordance with the present invention, applicants provide methods for treating cancer. In particular, the invention concerns treating cancers, such as recurrent or resistant cancers, for example, with an expression construct that affects regulation of expression of a specific nucleic acid sequence, and an agent that targets the expressed gene product of the specific nucleic acid sequence results in direct or indirect treatment of the cell.

More particularly, and in exemplary embodiments only, the invention relates to treating recurrent cancers with Erbitux™ and an expression construct encoding p53. Erbitux™ may be provide prior to, at the same time, or subsequent to p53 gene therapy. Thus, it is contemplated that time points for therapies will be as close as 4, 8, 12, or 24 hours. However, beneficial effects may be obtained (and perhaps with fewer side effects) with distinct times in the range of 3- to 6-months. Thus, the present invention contemplates times periods between p53 and Erbitux™ of 1, 2, 3, 4, 5, 6, 7, 8, 9, 10, 11, 12, 13 or 14 days, three, four, five, six, seven or eight weeks, one two, three four, five, or six months, and up to one year.

Various combinations may be employed, gene therapy is “A” and Erbitux™ is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The present invention may be utilized in a variety of solid cancers, such as brain cancer, head & neck cancer, esophageal cancer, tracheal cancer, lung cancer, liver cancer stomach cancer, colon cancer, pancreatic cancer, breast cancer, cervical cancer, uterine cancer, bladder cancer, prostate cancer, testicular cancer, skin cancer or rectal cancer. It also may be used against lymphomas or leukemias.

Local, region or systemic delivery of the exemplary p53 expression constructs and/or the exemplary Erbitux™ to patients is contemplated. It is proposed that this approach will provide clinical benefit, defined broadly as any of the following: reducing primary tumor size, reducing occurrence or size of metastasis, reducing or stopping tumor growth, inhibiting tumor cell division, killing a tumor cell, inducing apoptosis in a tumor cell, reducing or eliminating tumor recurrence.

Patients with unresectable tumors may be treated according to the present invention. As a consequence, the tumor may reduce in size, or the tumor vasculature may change such that the tumor becomes resectable. If so, standard surgical resection may be permitted.

A. Erbitux™

Erbitux™ (Cetuximab) is a recombinant, human/mouse chimeric monoclonal antibody that binds specifically to the extracellular domain of the human epidermal growth factor receptor (EGFR). Erbitux™ is composed of the Fv regions of a murine anti-EGFR antibody with human IgG1 heavy and kappa light chain constant regions and has an approximate molecular weight of 152 kDa. Erbitux™ is produced in mammalian (murine myeloma) cell culture.

Erbitux™ is a sterile, clear, colorless liquid of pH 7.0 to 7.4, which may contain a small amount of easily visible, white, amorphous, Cetuximab particulates. Each single-use, 50 ml vial contains 100 mg of Cetuximab at a concentration of 2 mg/ml and is formulated in a preservative-free solution containing 8.48 mg/ml sodium chloride, 1.88 mg/ml sodium phosphate dibasic heptahydrate, 0.42 mg/ml sodium phosphate monobasic monohydrate, and Water for Injection, USP. Erbitux™ binds specifically to the epidermal growth factor receptor (EGFR, HER1, c-ErbB-1) on both normal and tumor cells, and competitively inhibits the binding of epidermal growth factor (EGF) and other ligands, such as transforming growth factor-alpha. Binding of Erbitux™ to EGFR blocks phosphorylation and activation of receptor-associated kinases, resulting in inhibition of cell growth, induction of apoptosis, and decreased matrix metalloproteinase and vascular endothelial growth factor production. EGFR is a transmembrane glycoprotein that is a member of a subfamily of type I receptor tyrosine kinases including EGFR (HER1), HER2, HER3, and HER4. EGFR is constitutively expressed in many normal epithelial tissues, including the skin and hair follicle. Overexpression of EGFR is also detected in many human cancers including those of the colon and rectum.

In vitro assays and in vivo animal studies have shown that Erbitux™ inhibits the growth and survival of tumor cells that over-express the EGFR. No anti-tumor effects of Erbitux™ were observed in human tumor xenografts lacking EGFR expression. The addition of Erbitux™ to irinotecan or irinotecan plus 5-fluorouracil in animal studies resulted in an increase in anti-tumor effects compared to chemotherapy alone.

Erbitux™ administered as monotherapy or in combination with concomitant chemotherapy or radiotherapy exhibits nonlinear pharmacokinetics. The area under the concentration time curve (AUC) increased in a greater than dose proportional manner as the dose increased from 20 to 400 mg/m². Erbitux™ clearance (CL) decreased from 0.08 to 0.02 L/h/m2 as the dose increased from 20 to 200 mg/m², and at doses >200 mg/m2, it appeared to plateau. The volume of the distribution (Vd) for Erbitux™ appeared to be independent of dose and approximated the vascular space of 2-3 L/m². Following a 2-hour infusion of 400 mg/m² of Erbitux™, the maximum mean serum concentration (Cmax) was 184 μg/ml (range: 92-327 μg/ml) and the mean elimination half-life was 97 hours (range 41-213 hours). A 1 hr infusion of 250 mg/m² produced a mean Cmax of 140 μg/mL (range 120-170 μg/ml). Following the recommended dose regimen (400 mg/m² initial dose; 250 mg/m² weekly dose), Erbitux™ concentrations reached steady-state levels by the third weekly infusion with mean peak and trough concentrations across studies ranging from 168 to 235 and 41 to 85 μg/ml, respectively. The mean half-life was 114 hrs (range 75-188 hrs).

A population pharmacokinetic analysis was performed to explore the potential effects of selected covariates including race, gender, age, and hepatic and renal function on Erbitux™ pharmacokinetics. Female patients had a 25% lower intrinsic Erbitux™ clearance than male patients. Similar efficacy and safety were observed for female and male patients in the clinical trials; therefore, dose modification based on gender is not necessary. None of the other covariates explored appeared to have an impact on Erbitux™ pharmacokinetics. Erbitux™ has not been studied in pediatric populations.

The efficacy and safety of Erbitux™ alone or in combination with irinotecan were studied in a randomized, controlled trial (329 patients) and in combination with irinotecan in an open-label, single-arm trial (138 patients). Erbitux™ was further evaluated as a single agent in a third clinical trial (57 patients). Safety data from 111 patients treated with single agent Erbitux™ was also evaluated. All trials studied patients with EGFR-expressing metastatic colorectal cancer, whose disease had progressed after receiving an irinotecan-containing regimen.

A multicenter, randomized, controlled clinical trial was conducted in 329 patients randomized to receive either Erbitux™ plus irinotecan (218 patients) or Erbitux™ monotherapy (111 patients). In both arms of the study, Erbitux™ was administered as a 400 mg/m² initial dose, followed by 250 mg/m² weekly until disease progression or unacceptable toxicity. All patients received a 20-mg test dose on Day 1. In the Erbitux™ plus irinotecan arm, irinotecan was added to Erbitux™ using the same dose and schedule for irinotecan as the patient had previously failed. Acceptable irinotecan schedules were 350 mg/m² every 3 weeks, 180 mg/m² every 2 weeks, or 125 mg/m² weekly times four doses every 6 weeks. An Independent Radiographic Review Committee (IRC), blinded to the treatment arms, assessed both the progression on prior irinotecan and the response to protocol treatment for all patients. Of the 329 randomized patients, 206 (63%) were male. The median age was 59 years (range 26-84), and the majority was Caucasian (323, 98%). Eighty-eight percent of patients had baseline Kamofsky Performance Status. Fifty-eight percent of patients had colon cancer and 40% rectal cancer. Approximately two-thirds (63%) of patients had previously failed oxaliplatin treatment.

The efficacy of Erbitux™ plus irinotecan or Erbitux™ monotherapy was evaluated in all randomized patients. Analyses were also conducted in two pre-specified subpopulations: irinotecan refractory and irinotecan and oxaliplatin failures. The irinotecan refractory population was defined as randomized patients who had received at least two cycles of irinotecan-based chemotherapy prior to treatment with Erbitux™, and had independent confirmation of disease progression within 30 days of completion of the last cycle of irinotecan-based chemotherapy. The irinotecan and oxaliplatin failure population was defined as irinotecan refractory patients who had previously been treated with and failed an oxaliplatin-containing regimen.

The median duration of response in the overall population was 5.7 months in the combination arm and 4.2 months in the monotherapy arm. Compared with patients randomized to Erbitux™ alone, patients randomized to Erbitux™ and irinotecan experienced a significantly longer median time to disease progression.

Erbitux™, in combination with irinotecan, was studied in a single-arm, multicenter, open-label clinical trial in 138 patients with EGFR-expressing metastatic colorectal cancer who had progressed following an irinotecan containing regimen. Patients received a 20 mg test dose of Erbitux™ on day 1, followed by a 400 mg/m² initial dose, and 250 mg/m² weekly until disease progression or unacceptable toxicity. Patients received the same dose and schedule for irinotecan as the patient had previously failed. Acceptable irinotecan schedules were 350 mg/m² every 3 weeks or 125 mg/m² weekly times four doses every 6 weeks. Of 138 patients enrolled, 74 patients had documented progression to irinotecan as determined by an IRC. The overall response rate was 15% for the overall population and 12% for the irinotecan failure population. The median durations of response were 6.5 and 6.7 months, respectively. Erbitux™ was studied as a single agent in a multicenter, open-label, single-arm clinical trial in patients with EGFR-expressing metastatic colorectal cancer who progressed following an irinotecan-containing regimen. Of 57 patients enrolled, 28 patients had documented progression to irinotecan. The overall response rate was 9% for the all treated group and 14% for the irinotecan failure group. The median times to progression were 1.4 and 1.3 months, respectively. The median duration of response was 4.2 months for both groups.

Patients enrolled in the clinical studies were required to have immunohistochemical evidence of positive EGFR expression. Primary tumor or tumor from a metastatic site was tested with the DakoCytomation EGFR pharmDx™ test kit. Specimens were scored based on the percentage of cells expressing EGFR and intensity (barely/faint, weak to moderate, and strong). Response rate did not correlate with either the percentage of positive cells or the intensity of EGFR expression.

Erbitux™, used in combination with irinotecan, is indicated for the treatment of EGFR-expressing, metastatic colorectal carcinoma in patients who are refractory to irinotecan-based chemotherapy. Erbitux™ administered as a single agent is indicated for the treatment of EGFR-expressing, metastatic colorectal carcinoma in patients who are intolerant to irinotecan-based chemotherapy. The effectiveness of Erbitux™ is based on objective response rates. Currently, no data are available that demonstrate an improvement in disease-related symptoms or increased survival with Erbitux™.

The recommended dose of Erbitux™, in combination with irinotecan or as monotherapy, is 400 mg/m² as an initial loading dose (first infusion) administered as a 120 min IV infusion (maximum infusion rate 5 ml/min). The recommended weekly maintenance dose (all other infusions) is 250 mg/m² infused over 60 minutes (maximum infusion rate 5 ml/min). Premedication with an H1 antagonist (e.g., 50 mg of diphenhydramine IV) is recommended. Appropriate medical resources for the treatment of severe infusion reactions should be available during Erbitux™ infusions.

If the patient experiences a mild or moderate (Grade 1 or 2) infusion reaction, the infusion rate should be permanently reduced by 50%. Erbitux™ should be immediately and permanently discontinued in patients who experience severe (Grade 3 or 4) infusion reactions.

Erbitux™ is not to be administered as an IV push or bolus. Erbitux™ must be administered with the use of a low protein binding 0.22-401 micrometer in-line filter. Erbitux™ is supplied as a 50 ml, single-use vial containing 100 mg of Cetuximab at a concentration of 2 mg/ml in phosphate buffered saline. The solution should be clear and colorless and may contain a small amount of easily visible white amorphous Cetuximab particulates. Erbitux™ can be administered via infusion pump or syringe pump. Following the Erbitux™ infusion, a 1 hr observation period is recommended.

Erbitux™ (Cetuximab) is supplied as a single-use, 50 ml vial containing 100 mg of Cetuximab as a sterile, preservative-free, injectable liquid. Each carton contains one Erbitux™ vial (NDC 66733-948-23). Vials should be stored under refrigeration at 2° C. to 8° C. (36° F. to 46° F.). Increased particulate formation may occur at temperatures at or below 0° C. This product contains no preservatives. Preparations of Erbitux™ in infusion containers are chemically and physically stable for up to 12 hrs at 2° C. to 8° C. (36° F. to 46° F.) and up to 8 hrs at controlled room temperature (20° C. to 25° C.; 68° F. to 77° F.). Discard any remaining solution in the infusion container after 8 hrs at controlled room temperature or after 12 hrs at 2° C. to 8° C. Discard any unused portion of the vial.

U.S. Pat. No. 6,217,866 describes antibodies to EGFR and their uses. This document is hereby incorporated by reference in its entirety.

B. p53 Gene Therapy

Human p53 gene therapy has been described in the literature since the mid-1990's. Roth et al. (1996) reported on retroviral-based therapy, and Clayman et al. (1998) described adenoviral delivery. U.S. Pat. Nos. 6,017,524; 6,143,290; 6,627,190; 6,410,010; and 6,511,847, and U.S. Patent Application No. 2002/0077313 each describe methods of treating patients with p53, and are hereby incorporated by reference. Particular embodiments contemplate the use of the Ad-p53 vector Advexin™, more particularly in dosages amounts of about 10⁷, about 10⁸, about 10⁹, about 10¹⁰, about 10¹¹, about 10¹² and about 10¹³ viral particles, and any range derivable therein.

One particular mode of administration that can be used in conjunction with surgery is treatment of an operative tumor bed. Thus, in either the primary gene therapy treatment, or in a subsequent treatment, one may perfuse the resected tumor bed with the vector during surgery, and following surgery, optionally by inserting a catheter into the surgery site.

IX. Other Therapeutic Combinations

In accordance with the present invention, additional therapies may be applied with further benefit to the patients being treated with a combination of p53 gene therapy and Erbitux™. Such therapies include surgery, cytokines, toxins, drugs, dietary, or a non-p53-based gene therapy. Examples are discussed below.

A. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

1. Alkylating agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

a. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

b. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m 2/day or alternatively 0.4 mg/kg may be used for antineoplastic treatment. Treatment regimes are well know to those of skill in the art and can be found in the “Physicians Desk Reference” and in “Remington's Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation. Thus, it can be used in combination with troglitazone in the treatment of cancer.

c. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg//m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

d. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

e. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject

2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheisis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

a. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

In the present invention the inventors have employed troglitazone as an exemplary chemotherapeutic agent to synergistically enhance the antineoplastic effects of the doxorubicin in the treatment of cancers. Those of skill in the art will be able to use the invention as exemplified potentiate the effects of doxorubicin in a range of different pre-cancer and cancers.

b. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 min and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 years) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 Mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 Mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

c. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

d. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

e. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

4. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

a. Etoposide (VP16)

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

b. Taxol

Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

c. Vinblastine

Vinblastine is another example of a plant aklyloid that can be used in combination with troglitazone for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hrs. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m ², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

d. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can alos be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

e. Camptothecin

Camptothecin is an alkaloid derived from the chinese tree Camptotheca acuminata Decne. Camptothecin and its derivatives are unique in their ability to inhibit DNA Topoisomerase by stabilizing a covalent reaction intermediate, termed “the cleavable complex,” which ultimately causes tumor cell death. It is widely believed that camptothecin analogs exhibited remarkable anti-tumour and anti-leukaemia activity. Application of camptothecin in clinic is limited due to serious side effects and poor water-solubility. At present, some camptothecin analogs (topotecan; irinotecan), either synthetic or semi-synthetic, have been applied to cancer therapy and have shown satisfactory clinical effects. The molecular formula for camptothecin is C₂₀H₁₆N₂O₄, with a molecular weight of 348.36. It is provided as a yellow powder, and may be solubilized to a clear yellow solution at 50 mg/ml in DMSO 1N sodium hydroxide. It is stable for at least two years if stored at 2-8° C. in a dry, airtight, light-resistant environment.

5. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

a. Carmustine

Carmustine (sterile carmustine) is one of the nitrosoureas used in the treatment of certain neoplastic diseases. It is 1,3-bis(2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m² 50 mg/m² 60 mg/m² 70 mg/m² 80 mg/m² 90 mg/m² 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject.

b. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per ml). Lomustine is relatively insoluble in water (<0.05 mg per ml). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

6. Other Agents

Other agents that may be used include Avastin, Iressa® (gefitinib), Erbitux™, Tarceva® (erlotinib), Velcade, and Gleevec. In addition, growth factor inhibitors and small molecule kinase inhibitors have utility in the present invention as well. All therapies described in Cancer: Principles and Practice of Oncology Single Volume (Book with CD-ROM) by Vincent T. Devita (Editor), Samuel Hellman (Editor), Steven A. Rosenberg (Editor) Lippencott (2001), are hereby incorporated by reference. The following additional therapies are encompassed, as well.

a. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with Ad-mda7 gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

Tumor Necrosis Factor is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

b. Hormonal Therapy

The use of sex hormones according to the methods described herein in the treatment of cancer. While the methods described herein are not limited to the treatment of a specific cancer, this use of hormones has benefits with respect to cancers of the breast, prostate, and endometrial (lining of the uterus). Examples of these hormones are estrogens, anti-estrogens, progesterones, and androgens.

Corticosteroid hormones are useful in treating some types of cancer (lymphoma, leukemias, and multiple myeloma). Corticosteroid hormones can increase the effectiveness of other chemotherapy agents, and consequently, they are frequently used in combination treatments. Prednisone and dexamethasone are examples of corticosteroid hormones.

B. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques. Stereotactic radiotherapy is used to treat brain tumours. This technique directs the radiotherapy from many different angles so that the dose going to the tumour is very high and the dose affecting surrounding healthy tissue is very low. Before treatment, several scans are analysed by computers to ensure that the radiotherapy is precisely targeted, and the patient's head is held still in a specially made frame while receiving radiotherapy. Several doses are given.

Stereotactic radio-surgery (gamma knife) for brain tumors does not use a knife, but very precisely targeted beams of gamma radiotherapy from hundreds of different angles. Only one session of radiotherapy, taking about four to five hours, is needed. For this treatment you will have a specially made metal frame attached to your head. Then several scans and x-rays are carried out to find the precise area where the treatment is needed. During the radiotherapy, the patient lies with their head in a large helmet, which has hundreds of holes in it to allow the radiotherapy beams through.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

C. Subsequent Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

D. Gene Therapy

In another embodiment, the secondary treatment is a non-p53 gene therapy in which a second gene is administered to the subject. Delivery of a vector encoding p53 in conjuction with a second vector encoding one of the following gene products may be utilized. Alternatively, a single vector encoding both genes may be used. A variety of moleclues are encompassed within this embodiment, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors Rb, p16, MDA-7, PTEN and C-CAM are specifically contemplated.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

X. Pharmaceutical Compositions

According to the present invention, therapeutic compositions are administered to a subject. The phrases “pharmaceutically” or “pharmacologically acceptable” refer to compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions, vectors or cells of the present invention, its use in therapeutic compositions is contemplated. Supplementary active ingredients also can be incorporated into the compositions.

In various embodiments, agents that might be delivered may be formulated and administered in any pharmacologically acceptable vehicle, such as parenteral, topical, aerosal, liposomal, nasal or ophthalmic preparations. In certain embodiments, formulations may be designed for oral, inhalant or topical administration. In those situations, it would be clear to one of ordinary skill in the art the types of diluents that would be proper for the proposed use of the polypeptides and any secondary agents required.

Administration of compositions according to the present invention will be via any common route so long as the target tissue or surface is available via that route. This includes oral, nasal, buccal, respiratory, rectal, vaginal or topical. Alternatively, administration may be by intratumoral, intralesional, into tumor vasculature, local to a tumor, regional to a tumor, intradermal, subcutaneous, intramuscular, intraperitoneal or intravenous injection (systemic). Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. Routes of administration may be selected from intravenous, intrarterial, intrabuccal, intraperitoneal, intramuscular, subcutaneous, oral, topical, rectal, vaginal, nasal and intraocular.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In a particular embodiment, liposomal formulations are contemplated. Liposomal encapsulation of pharmaceutical agents prolongs their half-lives when compared to conventional drug delivery systems. Because larger quantities can be protectively packaged, this allows the opportunity for dose-intensity of agents so delivered to cells.

XI. Kits

Any of the compositions described herein may be comprised in a kit. In a non-limiting example, a p53 expression construct and/or Erbitux™, Iressa®(gefitinib), Tarceva® (erlotinib), or a mixture thereof may be comprised in a kit, although in particular aspects they are provided in separate contatiners. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for holding containers in close confinement for commercial sale. Such containers may include injection or blow-molded plastic containers into which the desired vials are retained.

### XII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell lines and and adenoviral vectors. The human non-small cell cancer cell line H1299 was kindly provided by Dr. A. Gazdar, Cancer Center Dallas, Tex. It is homozygously deleted for the p53 gene. It was grown in RPMI 1640 medium supplemented with 5% heat inactivated fetal bovine serum, glutamine, antibiotics and antimycotics. The human breast cancer cell line MDA-MB-468 was purchased from ATCC. These cells were cultured in high glucose Dulbecco's modified essential medium/F-12, supplemented with 10% heat inactivated fetal bovine serum, glutamine, antibiotics, and antimycotics. This cell line is heterogeneous for the mutated p53 gene and has a non-functional retinoblastoma (Rb) gene.

Two recombinant E1 deleted replication defective adenoviral vectors were used—Ad5CMV-LacZ (Ad-β-gal), and Ad5CMV-p53 (Ad-p53). The genes LacZ and p53 are under control of the human cytomegalovirus enhancer/promoter and an SV40 early polyadenylation signal replacing the E1 region of the adenovirus type 5 backbone (Zhang et al., 1994). The only difference between Ad5CMV-p53 and Ad5CMV-LacZ is that in Ad5CMV-p53 the E. coli LacZ gene was replaced by the human wild-type p53 sequence. (Zhang et al., 1993). The preparation of the viral stock was performed as previously described (Zhang et al., 1995). Briefly, a single clone of the Ad5CMV-p53 virus was prepared by plaque purification and amplified on a large scale in 293 cells, human embryonic kidney cells, which contain E1, needed by the adenovirus for replication. Infected 293 cells showing cytopathic effects were harvested, resuspended in phosphate-buffered saline (PBS), and lysed by three cycles of freezing and thawing to release the viral particles. The lysates were clear by centrifugation, the cell-free viral supernatants were mixed with saturated CsCl, and virus particles were banded by ultracentrifugation. The virus containing band was then collected and dialysed twice in PBS. The viral stocks were aliquoted and stored at −80° C. until use. The viral titers were determined by measurement of the optical density (OD). Before use the viral stock solutions were further diluted in PBS to the desired concentrations.

Proliferation assays. H1299 and MDA-MB-468 cells were seeded in 6-well plates or 24-well plates, respectively, at a density of 1×10⁴ cells/well. Next day the media was changed with a low serum medium of 0.5% and the cells were then transfected with Ad-p53 or Ad-β-gal at 0.5 MOI for the H1299 cells and 1 MOI for the MDA-MB-468 cells. Three days after transfection the cells were replenished with fresh media and transfected again with the same MOIs. This time, cetuximab (also known at least as C225) was added to obtain a final concentration of 30 nM. The treatment groups were Ad-β-gal, Ad-p53, or cetuximab alone, or combination of cetuximab with Ad-β-gal or Ad-p53. Cell counts were performed on day 3, 5, and 7 after the first treatment with cetuximab. On day 3 the cells were replenished with fresh media and cetuximab. The average of triplicate cell counts was calculated.

Western blot analysis. Cells were seeded in 10 cm dishes at 1×10⁶ cells/dish. Next day the cells were transfected with either Ad-p53 or Ad-β-gal at the above mentioned concentrations. The proteins were harvested daily after transfection using sodium dodecyl sulfate-polyacrylamide gel electrophoresis (SDS-PAGE) sample buffer. The lanes were loaded with equal amounts of protein as determined by BCA protein assay (Pierce, Rockford, Ill.). After electrophoresis at 100V for 90 min, the proteins were transferred from the gel to a Hybond-ECL membrane (Amersham International plc, Litte Chalfont, Buckinghamshire, England). After blocking with 5% dry milk supplemented with 0.3% Tween 20 (Sigma Chemical Co), the membranes were incubated with the primary antibodies. The following antibodies were used: p53, p21, EGFR, and p27. The membranes were developed according to Amersham's ECL western blotting protocol.

Cell cycle analysis by flow cytometry. Cells were seeded at 5×10⁵ cells in 60 mm dishes. Treatment and time course of treatment were identical to that used for the proliferation studies and the Western blots. On day four after the first Ad-p53 transduction, which corresponds to day 1 after cetuximab addition, trypsinized cells were washed once in PBS, fixed in 70% ethanol, and stored at 4° C. until use. The cells were incubated with 20 μg/ml propidium iodide (PI) and 20 μg/ml ribonuclease at 37° C. for 30 min. The analysis were performed with an EPICS profile II (Coulter Corporation, Hialeath, Fla.) equipped with an air-cooled argon ion laser emitting 488 nm at 15 mw. At least 10,000 events per sample were analysed and FITC fluorescence was collected using 525BP filter. For data analysis, the Coulter's Cytologic program was used. Mean peak fluorescence was determined for each histogram.

Terminal deoxynucleotidyl transferase-mediated dUTP-biotin nick end labeling (TUNEL) assay in situ for detction of apoptotic cells. The method of staining used was according to that previously described (Fujiwara et al., 1994). Briefly, cells were seeded in chamber slides (Becton Dickinson, Franklin Lakes, N.J.), and treated with combination of Ad-β-gal, or Ad-p53 with cetuximab at the same conditions as for cell cycle analysis. The slides were then fixed in 1:1 ethanol and aceton for 20 min in -20° C., the endogenous peroxidase was blocked with Methanol containing 3% H₂O₂ for 15 min at room temperature. The samples were incubated with 0.1% Triton X-100 and 0.1% sodium citrate at 4° C. for 2 min. For positive control 0.25 U/μl DNase was added for 20 minutes. The cells were then incubated with 0.1 U/μl terminal deoxynucleotidyl transferase (TdT) buffer (30 mM Tris (pH 7.2), 140 mM sodium cacodylate, and 1 mM cobalt chloride) and covered with 100 U/ml TdT (GIBCO BRL, Gaithersburg, Md.) 0.2 nM biotinylated 16-dUTP (Boehringer Mannheim, Indianapolis, Ind.) for 2 hrs at 37° C. The reaction was terminated by the transfering buffer (300 mM sodium chloride and 30 mM sodium citrate) for 30 min at room temperature. After a wash with PBS the slides were incubated with 2% bovine serum albumine for 10 min at room temperature. The slides were then incubated with 1:10 peroxydase conjugated streptavidin (DAKO Corp, Carpinteria, Calif.) for 30 min at room temperature, washed with PBS, and stained with diaminobenzidine (DAB) for 2 min at room temperature. The cells were counterstained with Hariris hematoxylin (Sigma, St. Louis, Mo.). Brown cells were considered apoptotic. Seven fields in every chamber were counted and the average percentage of brown cells was calculated.

DNA laddering. The cells were seeded, treated, and harvested analog to the method described for the cell cycle analysis. After one wash with PBS, the cells were resuspended in a buffer containing 0.5 μg/μl Proteinase K.

Immunoprecipitation and Kinase assay for Cdk2. Cells were seeded at a density of 1×10⁶ cells in 150 mm dishes. The treatment schedule was according to that used in the proliferation assay. The cells were treated according to the time schedule which was used for all experiments. After treatment, the cells were lysed and the proteins were harvested in a buffer containing 20 mM Tris-HCl pH 7.5, 150 mM NaCl, 1% NP40, and a cocktail of 10 different proteinase inhibitors. 500 μg of total cell proteins were used. After immunoprecipitation of Cdk2 using 5 μg anti Cdk2 antibody (sc.635, Santa Cruz) and 20 μl agarose conjugate, the pellet was washed 2 times in a buffer containing 20 mM Tris pH 7.5 and 10 mM MgCl₂. Per reaction 3 μg Histone H1, 5M unlabeled ATP and 10 μCi gamma ATP³² 400 Ci/mmol were added to the buffer and incubated 20 min at 37° C. The enzyme activity was inactivated by adding 2X SDS-gel loading buffer. After boiling the samples for 5 min, a SDS PAGE using a 12.5% gel was performed. The gel was then fixed in a solution containing 45% methanol and 10% acetic acid for 30 min. After drying in a gel dryer the gel was exposed to a film.

In vivo experiments. Four-to six-week-old female nude mice (nu/nu) were used for in vivo experiments. In brief, each mouse was first irradiated with 350rad from a Cs137 source to further suppress the immune system. Then, each irradiated mouse received 5×10⁶H1299 cells suspended in 200 μl phosphate-buffered saline (PBS) via subcutaneous injection into the lower back. When the resulting tumor grew to approximately 80 mm³ in a mouse, the mouse was randomized into one of six groups of 5 mice each. Cetuximab was injected intraperitoneally at a dose of 1 mg per mouse. Ad-p53 was injected at a dose of 5×10⁹ pfu intratumorally per mouse. Cetuximab was given on days 0, 3, 6, 9, 12, and 15; Ad-p53 was given on days 6 and 12. At day 21, a second treatment schedule was started. Cetuximab was injected on days 21, 24, 27, 30, 33, and 36, along with Ad-p53 on days 21, 27, and 33. Tumor size was measured every 3 days. When a mouse's tumor reached a volume of 500 mm³, the mouse was killed. When all mice in a group had been killed, the average tumor size for each group was calculated.

### Example 2

### Results

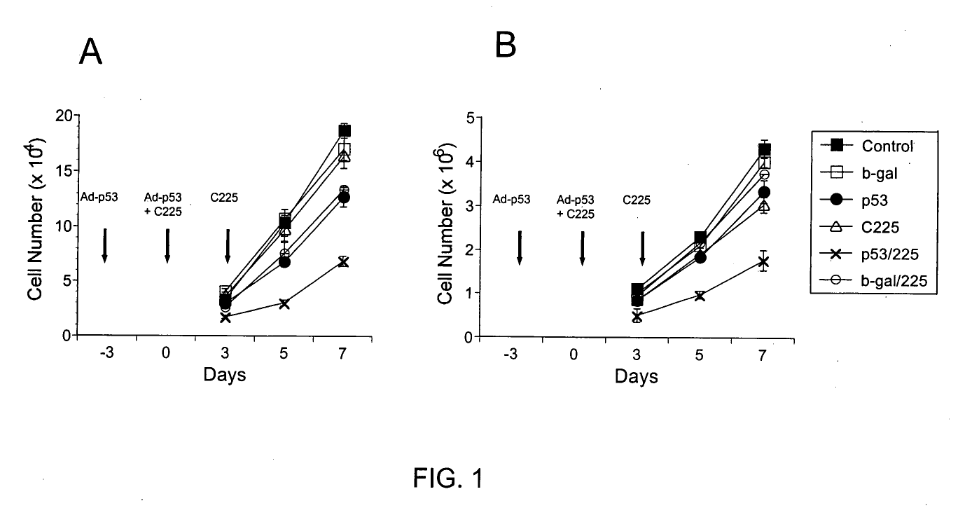

Additive growth inhibition after combined treatment with Ad-p53 and cetuximab was time dependent. When Ad-p53 and cetuximab were given in combination at the same time point at the concentrations mentioned in materials and methods, there was no significant additive growth inhibition compared to treatment with either Ad-p53 or cetuximab alone. However, an increasing additive growth inhibition was seen when cetuximab was added starting from day 0 to day three after Ad-p53 transduction (FIGS. 1A-1B). FIGS. 1A and 1B show two different experiments (proliferation assays) using H1299 where cetuximab was added for the first time three days after Ad-p53 transduction. A significant growth inhibition could be observed in the combination of Ad-p53 and cetuximab when compared to treatment with the single agents alone, or when cetuximab was combined with Ad-β-gal. The combination of Ad-p53 and cetuximab causes increasing growth inhibition when a delay of three days between Ad-p53 and cetuximab has taken place. When Ad-p53 and cetuximab were combined at the same time point, there was 13% more inhibition than the average of the growth inhibition of Ad-p53 or cetuximab alone. However, when cetuximab was added at later time points, there was an up to 40% enhanced growth inhibition in the combined treatment compared to Ad-p53 or cetuximab alone.

Induction of cell-cycle arrest or apoptosis after combined Ad-p53 and cetuximab treatment. Using Ad-p53 and cetuximab at the same time points and at the concentrations described in Example 1, they induced cell-cycle arrest in G1 and apoptosis in both cell lines. The percentage of apoptotic MDA-MB-468 cells increased and H1299 cells was increased above that seen with either cetuximab or Ad-p53 alone in three separate experiments. A representative experiment is shown in Table 3.

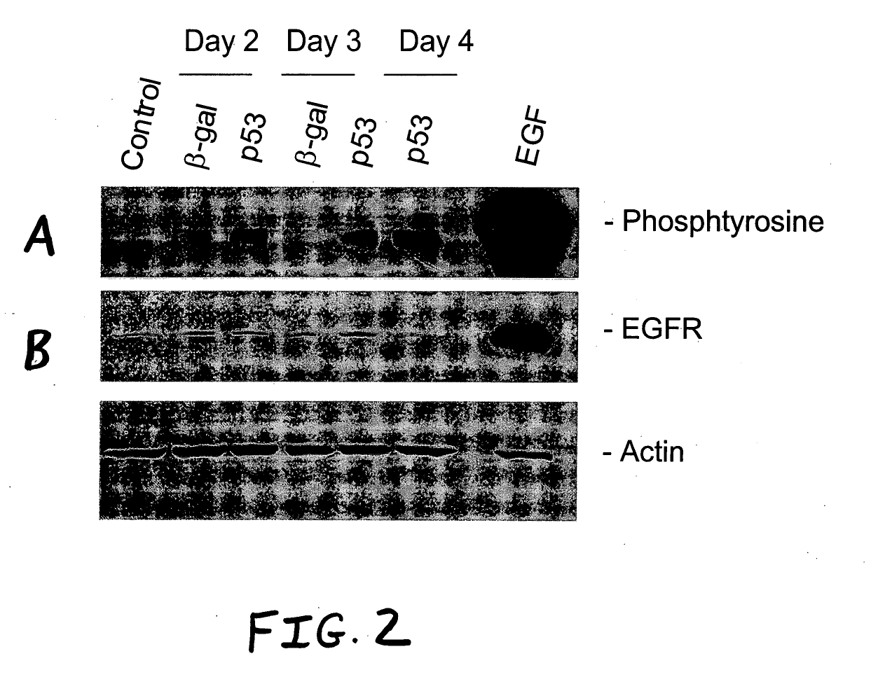

EGFR expression is highly induced by p53. After transfection with Ad-p53, the present inventors found a time-dependent upregulation of the EGF receptor. This induction had its maximum expression of seven-fold on day three after transduction compared to control untreated cells (FIG. 2A). Ad-β-gal did not have any effect on the expression of the EGF receptor as measured by western blot analysis. Since the EGF receptor has tyrosine kinase activity, the inventors were interested to know whether there was only an upregulation of the number or also a functional activation of the EGF receptor. Therefore, the present inventors stripped and reprobed the membranes for phosphotyrosine (FIG. 2B). The H1299 cell line showed an increase in the phosphorylation of the tyrosine kinase indicating a functional activation. In this cell line, however, there was no upregulation of the EGF receptor expression.

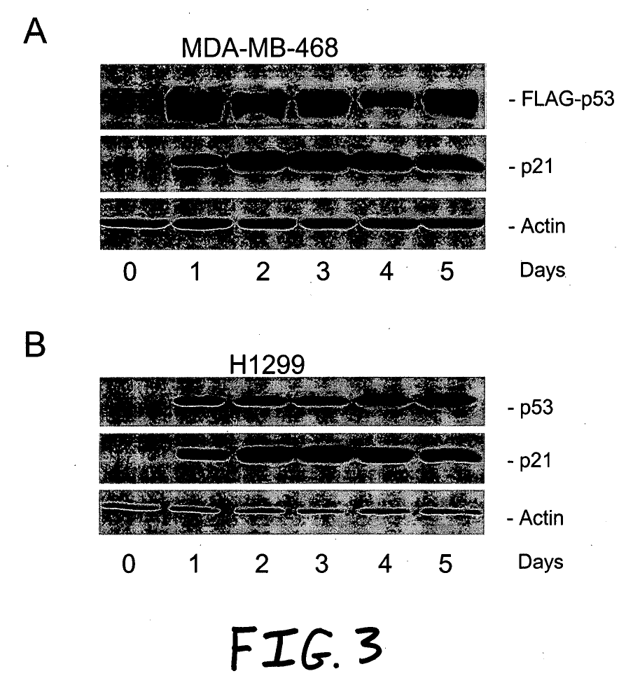

p53 expression and p21 induction. To obtain information about the time course of the EGF receptor expression, the present inventors transduced the p53-mutated cell line MDA-MB-468 with Ad-FLAGp53 and measured the expression of FLAGp53 over a five-day period by western blot (FIG. 3A). A gradual increase of FLAGp53 expression was observed with a peak expression on day two after transduction. P21^{WAF/CIP1} was also strongly induced by p53. FIG. 3B shows similar results for the H1299 cell line. In this p53 deleted cell line, Ad-p53 could be used for the p53 expression experiment.

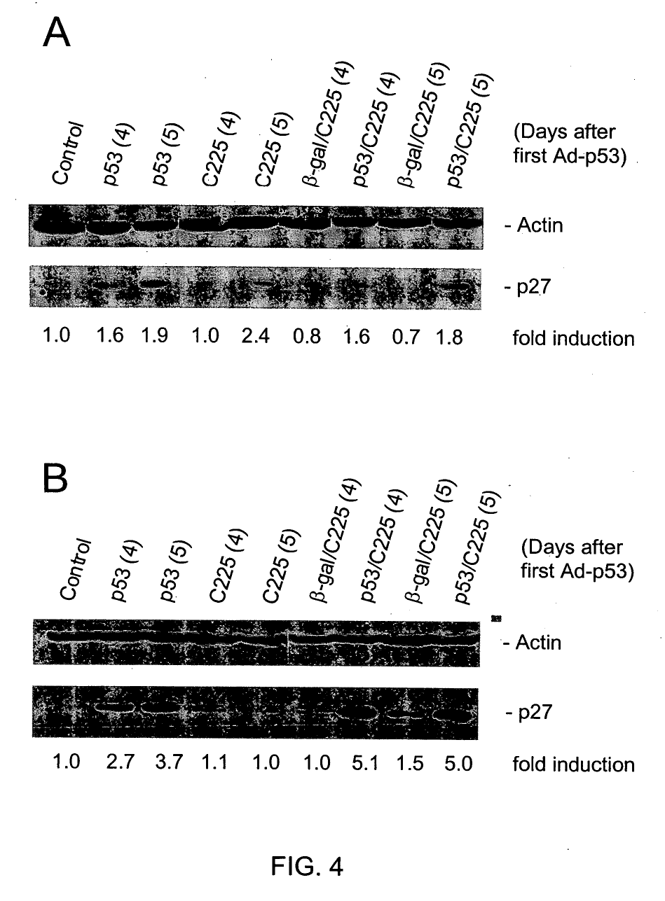

p53 and cetuximab upregulate p27. Cetuximab has been shown to be capable to induce p27 in certain cell lines. The inventors tested the combination of Ad-p53 and cetuximab in the system and found that also p53 can upregulate p27 (FIGS. 4A and 4B). No upregulation of the p27 protein could be detected by cetuximab alone. On the other hand, p53 alone induced p27 by 3.7-fold four days after transduction. When Ad-p53 was combined with cetuximab according to the protocol described in Material and Methods, a further increase of the p27 expression level to 5.1-fold could be observed. Ad-β-gal alone or in combination with cetuximab had no effect on the expression level of p27. In the MDA-MB-468 cell line, there was an 8-fold upregulated p27 expression two days after cetuximab treatment (FIG. 3A), there was also a two-fold p27 induction on day three after Ad-p53 transfection. However, the combination of Ad-p53 and cetuximab did not further enhance the p27 expression level. Cetuximab did not induce the expression of p21 in both cell lines, and there was no enhanced upregulation of p21 when cetuximab was combined with Ad-p53 compared to Ad-p53 alone (data not shown).

p21^{WAF1/CIP1} induces p27^{KIP1}. When MDA-MB-468 cells were transfected with Ad-p21 at 1 MOI, there was an upregulation of p₂₇^{KIP1} expression with a peak expression on day three after transduction. The maximum induction was two-fold. This induction appeared to be p53 independent.

Induced p27^{KIP1} induces G1-arrest. Cell cycle analysis was performed as described in Materials and Methods. When the cells were analysed after treatment with Ad-p53, Ad-β-gal, or cetuximab alone there was no significant change in the cell cycle phases in MDA-MB-468 cells. However, when Ad-p53 and cetuximab were combined an increase in the G1 phase from 55% to 71% was observed, indicating that the cells arrest in the G1 phase (Table 3). The number of apoptotic cells was also increased by 1.5-fold compared to Ad-p53 or cetuximab alone.

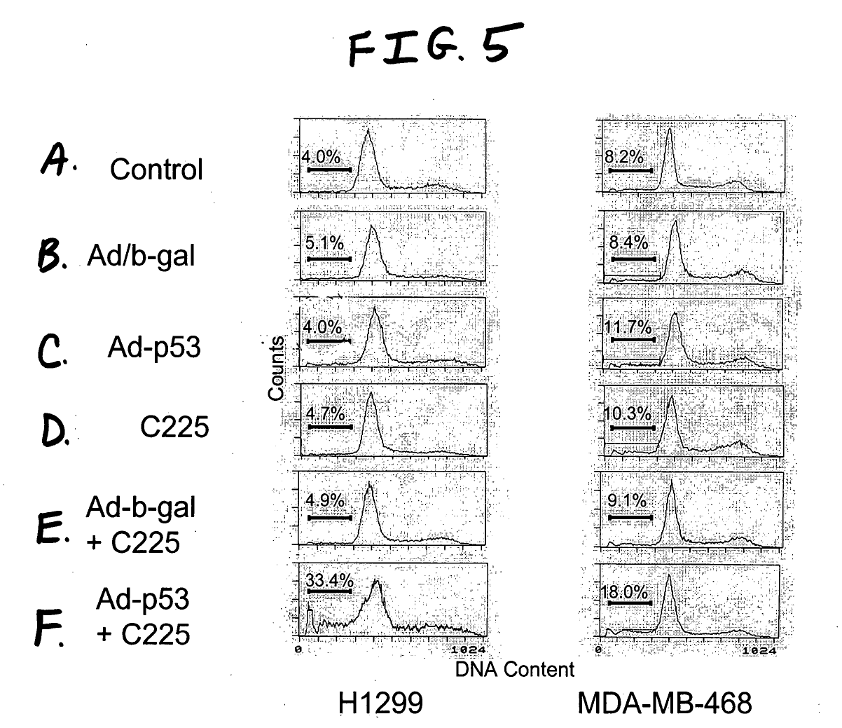

Overexpression of p27^{KIP1} induces apoptosis. H1299 cells did not change the fractions of the cell cycle, but there was a significant increase of the sub-G1 population of the cells from 5% to 33% (Table 4), which is indicative for fragmented DNA, a characteristic phenomenon in the process of apoptosis. Ad-β-gal alone or in combination did not have significant changes in the cell cycle or the number of apoptotic cells. TUNEL staining and DNA ladder confirm the data obtained by FACS analysis. FIGS. 5A-5F show the different treatment groups of H1299 cells in TUNEL staining. The combination of Ad-p53 and cetuximab had significantly more positive stained cells than all control groups. The level of apoptosis was less strong in MDA-MB-468 cells.

TABLE 3
468, Day 4

| | | | | | β-gal/ | P53/ |
|---|---|---|---|---|---|---|
| | Control | β-gal | P53 | cetuximab | cetuximab | cetuximab |
| G1 | 55.5 | 54.4 | 55.6 | 56.2 | 58.3 | 71.5 |
| S | 29 | 29.6 | 24.6 | 24.9 | 25.8 | 16.3 |
| G2/M | 15.5 | 16 | 19.8 | 19 | 15.9 | 12.2 |
| Apoptotic | 8.2 | 8.4 | 11.7 | 10.3 | 9.1 | 18 |
| cells | | | | | | |

TABLE 4
H1299, Day 4

| | | | | | β-gal/ | P53/ |
|---|---|---|---|---|---|---|
| | Control | β-gal | P53 | cetuximab | cetuximab | cetuximab |
| G1 | 66 | 68.1 | 64.7 | 69.7 | 66.8 | 61.1 |
| S | 20.7 | 19.2 | 19.4 | 20.6 | 20 | 28.9 |
| G2/M | 13.3 | 12.7 | 15.9 | 9.8 | 13.2 | 10 |
| Apoptotic | 4.0 | 5.1 | 8.4 | 4.7 | 4.9 | 33.4 |
| cells | | | | | | |

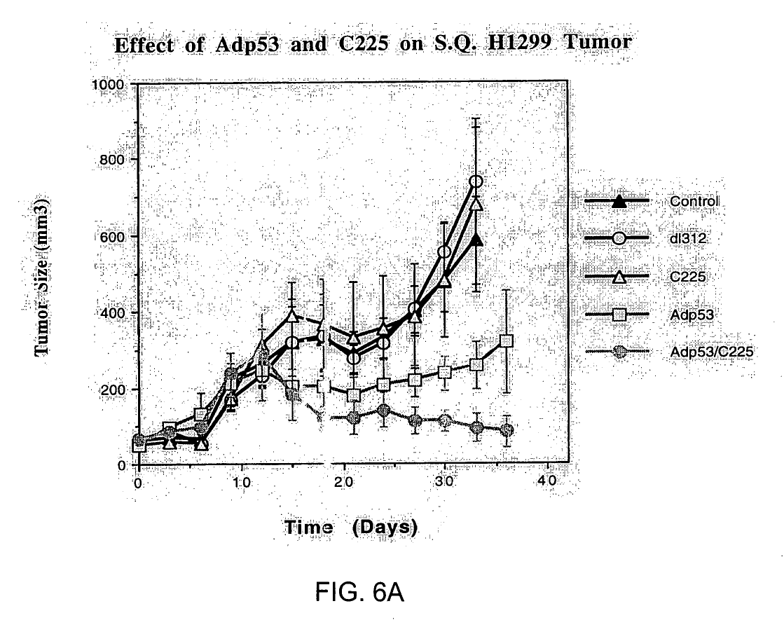

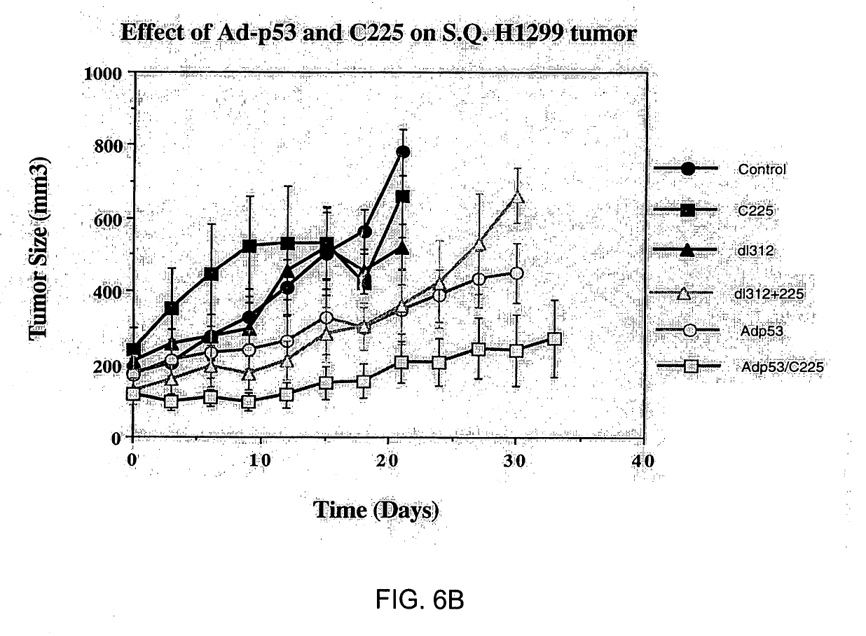

Inhibition of tumor growth in vivo after combined treatment with Ad-p53 and cetuximab. When H1299 cells were injected subcutaneously into nude mice as described in Example 1 and then injected with Ad-p53, cetuximab, or both. Ad-p53 alone inhibited tumor growth to some degree. Cetuximab alone had no effect on tumor growth. The combination of Ad-p53 and cetuximab, however, significantly inhibited tumor growth through 33 days of follow-up. The mice treated with cetuximab alone or with the controls PBS and d1312 were killed 21 days after treatment due to increased tumor size (FIGS. 6A-6B). A repeat of this experiment was performed twice with similar results.

### Example 3

### Significance of the Present Invention

In the present study, the inventors have shown that a combined therapy of cetuximab and Ad-p53 efficiently inhibits cell growth. Several different mechanisms seem to take place. Transduction of p53 results in induction of p21 and p27“ ”, both of which are known as universal inhibitors of the cyclin dependent kinases with resulting growth inhibition. The very low concentrations of Ad-p53 as used in this study did not an effect on growth of tumor cells. Cetuximab also induces p27^{KIP1}, but not p21. This also resulted in growth inhibition by cell cycle arrest.

The combination of Ad-p53 and cetuximab enhanced the growth inhibitory effect compared to treatment with either Ad-p53 or cetuximab alone. A possible explanation for this phenomenon may be different mechanisms. Both Ad-p53 and cetuximab have different pathways to inhibit growth. Another possible mechanism is the block of the EGF receptor by cetuximab, which has been upregulated by p53. In this system, p53-induced transduction of EGF receptor which would counteract the ggrowth inhibition by p53. Using cetuximab, this counteraction would be abrogated by blocking the growth factor dependent cell growth. A third possible mechanism may be the induction of p27^{KIP1}. In H1299 cells, which have low levels of endogeneous EGF receptor, cetuximab has an effect on its own, and p27^{KIP1} is not induced. However, in combination with p53 which induced p27^{KIP1} on its own, p27^{KIP1} could be further upregulated. This indicates that p53 makes the cells more sensitive to cetuximab, either through induction of EGF receptor, which offers more target receptors, or through a not yet identified downstream mechanism.

### Example 4

### Exemplary Treatment of the Invention

An individual having cancer, which may be metastatic cancer and/or cancer refractory to one or more cancer treatments, for example, is in need of therapy. Cancer cells are obtained from the individual. An expression construct is generated using standard molecular biology techniques known in the art, for example, wherein the construct comprises a sequence capable of affecting expression of another nucleic acid sequence, wherein affected nucleic acid sequence encodes a gene product that is targetable. The expression construct is delivered to one or more of the cancer cells, and the expression profile of one or more genes is determined, such as by analyzing a gene expression microchip standard in the art. A particular expression construct-responsive nucleic acid sequences is identified that encodes a gene product, and the gene product is known to have an agent that targets it or may have an agent that is produced to target the gene product.

Following identification of the suitable expression construct and agent, the compositions are delivered to the individual in need thereof, and/or are delivered to other individuals in need thereof having the same or similar cancers. Delivery regimes are optimized, such as by standard and routine pharmaceutical methods in the art.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XIII. REFERENCES

The following references, to the extent that they provide exemplary procedural or other details supplementary to those set forth herein, are specifically incorporated herein by reference:
U.S. Pat. No. 4,659,774
U.S. Pat. No. 4,659,774
U.S. Pat. No. 4,682,195
U.S. Pat. No. 4,682,195
U.S. Pat. No. 4,683,202
U.S. Pat. No. 4,683,202
U.S. Pat. No. 4,797,368
U.S. Pat. No. 4,797,368
U.S. Pat. No. 4,816,571
U.S. Pat. No. 4,816,571
U.S. Pat. No. 4,879,236
U.S. Pat. No. 4,879,236
U.S. Pat. No. 4,959,463
U.S. Pat. No. 4,959,463
U.S. Pat. No. 5,139,941
U.S. Pat. No. 5,139,941
U.S. Pat. No. 5,141,813
U.S. Pat. No. 5,141,813
U.S. Pat. No. 5,264,566
U.S. Pat. No. 5,264,566
U.S. Pat. No. 5,302,523
U.S. Pat. No. 5,302,523
U.S. Pat. No. 5,322,783
U.S. Pat. No. 5,322,783
U.S. Pat. No. 5,384,253
U.S. Pat. No. 5,384,253
U.S. Pat. No. 5,428,148
U.S. Pat. No. 5,428,148
U.S. Pat. No. 5,464,765
U.S. Pat. No. 5,464,765
U.S. Pat. No. 5,538,877
U.S. Pat. No. 5,538,877
U.S. Pat. No. 5,538,880
U.S. Pat. No. 5,538,880
U.S. Pat. No. 5,550,318
U.S. Pat. No. 5,550,318
U.S. Pat. No. 5,554,744
U.S. Pat. No. 5,554,744
U.S. Pat. No. 5,563,055
U.S. Pat. No. 5,563,055
U.S. Pat. No. 5,574,146
U.S. Pat. No. 5,574,146
U.S. Pat. No. 5,580,859
U.S. Pat. No. 5,580,859
U.S. Pat. No. 5,589,466
U.S. Pat. No. 5,589,466
U.S. Pat. No. 5,602,244
U.S. Pat. No. 5,602,244
U.S. Pat. No. 5,610,042
U.S. Pat. No. 5,610,042
U.S. Pat. No. 5,645,897
U.S. Pat. No. 5,645,897
U.S. Pat. No. 5,656,610
U.S. Pat. No. 5,656,610
U.S. Pat. No. 5,670,488
U.S. Pat. No. 5,670,488
U.S. Pat. No. 5,677,178
U.S. Pat. No. 5,677,178
U.S. Pat. No. 5,702,932
U.S. Pat. No. 5,702,932
U.S. Pat. No. 5,705,629
U.S. Pat. No. 5,705,629
U.S. Pat. No. 5,736,524
U.S. Pat. No. 5,736,524
U.S. Pat. No. 5,747,469
U.S. Pat. No. 5,747,469
U.S. Pat. No. 5,747,869
U.S. Pat. No. 5,747,869
U.S. Pat. No. 5,780,448
U.S. Pat. No. 5,780,448
U.S. Pat. No. 5,789,215
U.S. Pat. No. 5,789,215
U.S. Pat. No. 5,871,986
U.S. Pat. No. 5,871,986
U.S. Pat. No. 5,879,703
U.S. Pat. No. 5,879,703
U.S. Pat. No. 5,932,210
U.S. Pat. No. 5,932,210
U.S. Pat. No. 5,945,100
U.S. Pat. No. 5,945,100
U.S. Pat. No. 5,981,225
U.S. Pat. No. 5,981,225
U.S. Pat. No. 5,981,274
U.S. Pat. No. 5,981,274
U.S. Pat. No. 5,994,136
U.S. Pat. No. 5,994,624
U.S. Pat. No. 5,994,624
U.S. Pat. No. 6,013,516
U.S. Pat. No. 6,013,516
U.S. Pat. No. 6,017,524
U.S. Pat. No. 6,017,524
U.S. Pat. No. 6,069,134
U.S. Pat. No. 6,069,134
U.S. Pat. No. 6,136,594
U.S. Pat. No. 6,136,594
U.S. Pat. No. 6,143,290
U.S. Pat. No. 6,143,290
U.S. Pat. No. 6,143,290
U.S. Pat. No. 6,143,290
U.S. Pat. No. 6,210,939
U.S. Pat. No. 6,210,939
U.S. Pat. No. 6,217,866
U.S. Pat. No. 6,296,845
U.S. Pat. No. 6,296,845
U.S. Pat. No. 6,410,010
U.S. Pat. No. 6,410,010
U.S. Pat. No. 6,410,010
U.S. Pat. No. 6,410,010
U.S. Pat. No. 6,511,184
U.S. Pat. No. 6,511,184
U.S. Pat. No. 6,511,847
U.S. Pat. No. 6,511,847
U.S. Pat. No. 6,627,190
U.S. Pat. No. 6,627,190
U.S. Appln. 2002/0006914
U.S. Appln. 2002/0006914
U.S. Appln. 2002/0077313
U.S. Appln. 2002/0077313
U.S. Appln. 2002/0028785
U.S. Appln. 2002/0028785
Aksentijevich et al., Hum. Gene Ther., 7(9):1111-1122, 1996.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Bakhshi et al., Cell, 41(3):899-906, 1985.
Bargonetti et al., Cell, 65(6):1083-1091, 1991.
Bittner et al., Methods in Enzymol, 153:516-544, 1987.
Blomer et al., J. Virol., 71(9):6641-6649, 1997.
Casey et al., Oncogene, 6(10):1791-1797, 1991.
Chen and Okayama, Mol. Cell Biol., 7(8):2745-2752, 1987.
Clayman et al. 1995b
Clayman et al. J. Clin. Oncol., 16(6):2221-2232, 1998.
Clayman et al., Cancer Res., 55(14):1-6, 1995.
Cleary and Sklar, Proc. Natl. Acad. Sci. USA, (21):7439-7443, 1985.
Cleary et al., J. Exp. Med., 164(1):315-320, 1986.
Cotten et al., Proc. Natl. Acad. Sci. USA, 89(13):6094-6098, 1992.
Couch et al., Am. Rev. Resp. Dis., 88:394403, 1963.
Coupar et al., Gene, 68:1-10, 1988.
Curiel, Nat. Immun., 13(2-3):141-164, 1994.
Doyle, Semin. Oncol., 20(4):326-337, 1993.
Fechheimer, et al., Proc Natl. Acad. Sci. USA, 84:8463-8467, 1987.
Felgner et al., Proc. Natl. Acad. Sci. USA, 84(21):7413-7417, 1987.
Fields and Jang, Science, 249(4972):1046-1049, 1990.
Fraley et al., Proc. Natl. Acad. Sci. USA, 76:3348-3352, 1979.
Friedmann, Science, 244:1275-1281, 1989.
Froehler et al., Nucleic Acids Res., 14(13):5399-5407, 1986.
Gabizon et al., Cancer Res., 50(19):6371-6378, 1990.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gomez-Foix et al., J. Biol. Chem., 267:25129-25134, 1992.
Gopal, Mol. Cell Biol., 5:1188-1190, 1985.
Graham and Prevec, Biotechnology, 20:363-390, 1992.
Graham and Van Der Eb, Virology, 52:456-467, 1973.
Graham et al, J. General Virology, 36:59-74, 1977.
Grunhaus and Horwitz, Seminar in Virology, 3:237-252, 1992.
Harland and Weintraub, J. Cell Biol., 101(3):1094-1099, 1985.
Herz and Gerard, Proc. Natl. Acad. Sci. USA, 90:2812-2816, 1993.
Herz and Roizman, Cell, 33(1):145-151, 1983.
Hollstein et al., Science, 253(5015):49-53, 1991.
Horwich et al. J. Virol., 64:642-650, 1990.
Kaeppler et al., Plant Cell Reports, 9:415-418, 1990.
Kaneda et al., Science, 243:375-378, 1989.
Kato et al, J. Biol. Chem., 266:3361-3364, 1991.
Kelleher and Vos, Biotechniques, 17(6): 1110-7, 1994.
Kerr et al., Br. J. Cancer, 26(4):239-257, 1972.
Laughlin et al., J. Virol., 60(2):515-524, 1986.
Le Gal La Salle et al., Science, 259:988-990, 1993.
Lebkowski et al., Mol. Cell. Biol., 8(10):3988-3996, 1988.
Levrero et al., Gene, 101:195-202, 1991.
Liu et al., Cancer Res., 55(14):3117-3122, 1995.
Macejak and Sarnow, Nature, 353:90-94, 1991.
Mann et al., Cell, 33:153-159, 1983.
Martin et al., Nature, 345(6277):739-743, 1990.
McLaughlin et al., J. Virol., 62(6):1963-1973, 1988.
Mercer, Critic. Rev. Eukar. Gene Express. 2:251-263, 1992.
Mietz et al., EMBO J., 11(13):5013-5020, 1992.
Miller et al., Am. J. Clin. Oncol., 15(3):216-221, 1992.
Muzyczka, Curr. Topics Microbiol. Immunol., 158:97-129, 1992.
Nabel et al., Science, 244(4910):1342-1344, 1989.
Naldini et al., Science, 272(5259):263-267, 1996.
Nicholas and Shenk, Cell, 17:683-689, 1979.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Paskind et al., Virology, 67:242-248, 1975.
PCT Appln. WO 99/18933
PCT Appln. WO 94/09699
PCT Appln. WO 95/06128
PCT Appln. WO 98/07408
Pelletier and Sonenberg, Nature, 334(6180):320-325, 1988.
Philip et al., J. Biol. Chem., 268(22):16087-16090, 1993.
   Physicians Desk Reference
Potter et al., Proc. Natl. Acad. Sci. USA, 81:7161-7165, 1984.
Racher et al., Biotechnology Techniques, 9:169-174, 1995.
Ragot et al., Nature, 361:647-650, 1993.
Remington's Pharmaceutical Sciences, 15^{th} ed., pages 1035-1038 and 1570-1580, Mack Publishing Company, Easton, Pa., 1980.
Renan, Radiother. Oncol., 19:197-218, 1990.
Rich et al., Hum. Gene Ther., 4:461-476, 1993.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Rosenfeld et al., Science, 252:431-434, 1991.
Rosenfeld, et al., Cell, 68:143-155, 1992.
Roth et al., Nat Med., 2(9):985-991, 1996.
Roux et al., Proc. Natl. Acad. Sci. USA, 86:9079-9083, 1989.
Sambrook et al., In: Molecular cloning, Cold Spring Harbor Laboratory Press, Cold Spring Harbor, N.Y., 2001.
Shaw et al., Proc. Natl. Acad. Sci. USA, 89(10):4495-4499, 1992.
Sheikh et al., Oncogene, 15:1095-1101, 1997.
Smith and Rutledge, Natl. Cancer Inst. Monogr., 42:141-143, 1975.
Solodin et al., Biochemistry, 34(41):13537-13544, 1995.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Swisher et al., Proc. Am. Soc. Clin. Oncol., 16:437a, 1997.
Temin, In: Gene Transfer, Kucherlapati (Ed.), NY, Plenum Press, 149-188, 1986.
Templeton et al., Nat. Biotechnol., 15(7):647-652, 1997.
Thierry et al., Proc. Natl. Acad. Sci. USA, 92(21):9742-9746, 1995.
Theodorescu et al., Canc. Res., 51:4486-4491, 1991.
Top et al., J. Infect. Dis., 124:155-160, 1971.
Tratschin et al., Mol. Cell. Biol., 4:2072-2081, 1984.
Tsujimoto and Croce, Proc. Natl. Acad. Sci. USA, 83(14):5214-5218, 1986.
Tsujimoto et al., Science, 228(4706):1440-1443, 1985.
Tsukamoto et al., Nat. Genet., 9(3):243-248, 1995.
Tur-Kaspa et al., Mol. Cell Biol., 6:716-718, 1986.
Wang et al., J. Biol. Chem., 276(47):43604-43610, 2001.
Weinberg, Science, 254(5035):1138-1146, 1991.
Wilcock and Lane, Nature, 349(6308):429-431, 1991.
Wilder et al., Cancer Res., 59:410-413, 1999a.
Wilder et al., Gene Therapy, 6:57-62, 1999b.
Wilson et al., Science, 244:1344-1346, 1989.
Wong et al., Gene, 10:87-94, 1980.
Wu and Wu, Biochemistry, 27:887-892, 1988.
Wu and Wu, J. Biol. Chem., 262:4429-4432, 1987.
Yang and Huang, Gene Therapy, 4 (9):950-960, 1997.
Yonish-Rouach et al., Nature, 352(6333):345-347, 1991.
Young et al., N Engl J Med. 7:299(23):1261-1266, 1978.
Yu et al. Proc. Natl. Acad. Sci. USA 96(25):14517-14522, 1999.
Yu et al. World J. Gasteroenterol. 10(13):1979-1983, 2004.
Zakut-Houri et al., EMBO J., 4(5):1251-1255, 1985.
Zhang et al., Biofeedback, 15(5) 868-872, 1993.
Zhang et al., Cancer Gene Ther., 1:5-13, 1994.
Zhang et al., Human Gene Ther. 6:155-164, 1995.
Zhao et al., Genes Devel. 14:981-993, 2000.
Zhu et al., Science, 261(5118):209-211, 1993.
Zufferey et al., Nat. Biotechnol., 15(9):871-875, 1997.

What is claimed is:

1. A method of treating a subject with cancer comprising administering to said subject:
(a) a p53 expression construct comprising a nucleic acid segment encoding p53, said segment under the control of a promoter active in a cancer cell of said subject, said p53 expression construct expressing p53 in said cancer cell; and
(b) an agent that targets the expressed product of a p53 expression construct-responsive nucleic acid sequence in said cell, whereby said expression construct and agent are provided in amounts that treat said cancer.

2. The method of claim 1, wherein the expression construct and the agent are administered concomitantly or in succession.

3. The method of claim 1, wherein the expression construct is administered prior to the agent.

4. The method of claim 1, wherein the agent is further defined as targeting the expressed product of a p53 expression construct-responsive nucleic acid sequence or a downstream nucleic acid sequence therefrom.

5. The method of claim 1, wherein the p53-responsive nucleic acid sequence is upregulated in response to the p53 expression construct.

6. The method of claim 1, wherein the p53-responsive nucleic acid sequence is downregulated in response to the p53 expression construct.

7. The method of claim 1, wherein the p53-responsive nucleic acid sequence encodes a growth factor receptor, a receptor tyrosine kinase, a cell surface receptor, or a combination thereof.

8. The method of claim 1, wherein the p53-responsive nucleic acid sequence is epidermal growth factor receptor (EGFR), cox-2, Bax, IGFBP3, IGFBP4, Mdm2, MMP-2, TGF-alpha, IL-8R, p21, caveolin, DR5, Jun-B, Gpc, HO1, Btk, Htk, Bax, PIG3, Mdm2, PET-3, PET-7, ATDC, B61, PET-1, PET-8, PET-4, Gadd45, RTP, IGBP3, ET2, Mat8, PET-9, Fas, PET-11, 14-3-3σ, endothelin 2, negative growth regulator MyD118, TRAIL receptor 2 (KILLER/DR5), TGF-beta superfamily protein, caveolin, collagen, type II alpha1, nuclear matrix protein NRP/B(NRPB), p53, GADD45, regulator of G-protein signaling 2 G0S8, potassium channel alpha subunit, tyrosine protein kinase receptor eck, Pig3, actin alpha 2, APO-1/FAS, cystathionin-beta-synthase, macrophage stimulating protein (msp), complement component 4A, tetraspan NET-1, keratin 5, plasminogen activator inhibitor PAI-1, Ral GDP dissociation stimulator, collagen type VI alpha 1, TGF-alpha, endogenous retrovirus H and E sequence, keratin 17, EST similar to KIAA0835 protein, keratin 19, rhoHP 1, serum amyloid a protein precursor, interferon-induced 17-kD protein, interleukin-2 receptor beta chain (IL-2Rb), annexin-XIII, semaphorin V, neutrophil NADPH oxidase 2, estradiol 17 beta dehydrogenase 1, BMP4, P protein (melanocyte-specific transporter), thrombospondin 1, human activated p21cdc42Hs kinase (ack), P2XM, Pig12, phosphoglyverate mutase I, possible GTP-binding protein hsr1, superoxide dismutase 3 (extracellular), cyclin-dependent kinase 2, retinoblastoma-binding protein, DNA replication licensing factor cdc47 homolog, prothymosin alpha, EST similar to cyclin B2, ADP-ribosylation factor-like protein 2 (ARL2), human non-histone chromosomal protein HMG-17, mitotic feedback control protein Madp2 homology, KIAA0101 gene, HMG2, KIAA0030, prostatic binding protein, ATPase Na+/K+ transporting beta 1 polypeptide, lamin B receptor, DNA primase polypeptide 1, topoisomerase (DNA) II alpha, Myb proto-oncogene protein, human effector cell protease receptor-1 (EPR-1), CCAAT/enhancer binding protein C/EPBalpha, ribosomal protein S6 kinase 90 kD polypeptide 2, and InsP3 5 phosphatase, component C1 inhibitor, catechol o-methyltransferase, L-histidine decarboxylase, carboxyl ester lipase, interleukin 8 receptor-alpha, alpha-fetoprotein, alpha 1-acid glyprotein 2, cardiotrophin, IGFBP4, JunB, myoglobin, or MDR1.

9. The method of claim 1, wherein the agent is a small molecule or an antibody.

10. The method of claim 9, wherein the antibody is a monoclonal antibody.

11. The method of claim 1, wherein the p53-responsive nucleic acid sequence is EGFR and the agent is cetuximab, gefitinib, or erlotinib.

12. The method of claim 1, wherein said expression construct is a viral expression construct.

13. The method of claim 12, wherein said viral expression construct is a retroviral construct, a herpesviral construct, an adenoviral construct, an adeno-associated viral construct, or a vaccinia viral construct.

14. The method of claim 12, wherein said viral expression construct is a replication-competent virus.

15. The method of claim 12, wherein said viral expression construct is a replication-defective virus.

16. The method of claim 1, wherein said expression construct is a non-viral expression construct.

17. The method of claim 16, wherein said non-viral expression construct is comprised within a lipid vehicle.

18. The method of claim 1, wherein said promoter is selected from CMV IE, RSV LTR, β-actin, Ad-E1, Ad-E2 or Ad-MLP.

19. The method of claim 1, further defined as indirectly or directly producing apoptosis in said cancer cell.

20. The method of claim 1, further defined as conferring chemosensitivity to said cancer cell.

21. A method of treating a subject with cancer comprising administering to said subject, in combination,
(a) an expression construct comprising a nucleic acid segment encoding p53, said segment under the control of a promoter active in a cancer cell of said subject, said expression construct expressing p53 in said cancer cell; and
(b) cetuximab,
whereby said expression construct and cetuximab are provided in amounts that treat said cancer.

22. The method of claim 21, wherein said cancer is selected from the group consisting of brain cancer, head & neck cancer, esophageal cancer, tracheal cancer, lung cancer, liver cancer stomach cancer, colon cancer, pancreatic cancer, breast cancer, cervical cancer, uterine cancer, bladder cancer, prostate cancer, testicular cancer, skin cancer, rectal cancer lymphoma and leukemia.

23. The method of claim 21, wherein the cancer is metastatic.

24. The method of claim 21, wherein cancer is recurrent.

25. The method of claim 24, wherein recurrence is recurrence at a primary tumor site.

26. The method of claim 24, wherein recurrence is recurrence at a metastatic site.

27. The method of claim 21, wherein said subject has had surgical resection prior to administration (a).

28. The method of claim 21, further comprising surgical resection following administration (b).

29. The method of claim 21, wherein administration (a) is selected from the group consisting of intratumoral, to a tumor vasculature, local to a tumor, regional to a tumor, and systemic.

30. The method of claim 21, wherein administration (b) is selected from the group consisting of intratumoral, to a tumor vasculature, local to a tumor, regional to a tumor, and systemic.

31. The method of claim 21, wherein the subject is a human subject.

32. The method of claim 21, further comprising an additional distinct cancer therapy.

33. The method of claim 32, wherein the additional distinct cancer therapy is chemotherapy, radiotherapy, non-p53 gene therapy, non-Erbitux™ immunotherapy, hormonal therapy, or toxin therapy.

34. A pharmaceutical formulation comprising (a) an expression construct comprising a nucleic acid segment encoding p53, said segment under the control of a promoter active in a cancer cell of said subject; and (b) an agent that targets a gene product of the nucleic acid segment.

35. A kit comprising, in separate containers, (a) an expression construct comprising a nucleic acid segment encoding p53, said segment under the control of a promoter active in a cancer cell of said subject; and (b) an agent that targets a gene product of the nucleic acid segment.
